(19) United States
(12) Patent Application Publication
(10) Pub. No.: US 2005/0170345 A1
(43) Pub. Date: Aug. 4, 2005

(54) Neuroglobin is up-regulated by and protects neurons from hypoxic-ischemic injury

(76) Inventors: Yunjuan Sun, Novato, CA (US); Kunlin Jin, Novato, CA (US); Xiao Ou Mao, Novato, CA (US); Yonghua Zhu, Novato, CA (US); David A. Greenberg, Sonoma, CA (US)

Correspondence Address: QUINE INTELLECTUAL PROPERTY LAW GROUP, P.C., P O BOX 458, ALAMEDA, CA, 94501, US

(21) Appl. No.: 10/494,554

(22) PCT Filed: Nov. 6, 2002
(86) PCT No.: PCT/US02/35575

Related U.S. Application Data
(60) Provisional application No. 60/337,701, filed on Nov. 7, 2001.
(60) Provisional application No. 60/375,519, filed on Apr. 24, 2002.

(51) Int. Cl. C12Q 1/68; G01N 33/53; G01N 33/567

(52) U.S. Cl. 435/6; 435/7.2

(57) ABSTRACT

This invention pertains to the discovery that neuroglobin (Ngb) expression helps promote neuronal survival from hypoxic-ischemic insults. Neuroglobin thus provides a good target to screen for agents that mitigate harmful effects from hypoxic-/ischemic insult. Methods are provided for screening for agents that promote neuronal survival from hypoxic-ischemic insult (e.g., ischaemic injury such as caused by myocardial infarction, stroke induced neuron death, reperfusion injury, traumatic head injury, cardiac arrest, asphyxiation, and the like).

### CROSS-REFERENCE TO RELATED APPLICATIONS

This application claims priority to and benefit of U.S. Ser. No. 60/337,710, filed on Nov. 6, 2001, and U.S. Ser. No. 60/375,519, filed on Apr. 24, 2002, both of which are incorporated herein by reference in their entirety for all purposes.

### STATEMENT AS TO RIGHTS TO INVENTIONS MADE UNDER FEDERALLY SPONSORED RESEARCH AND DEVELOPMENT

This work was supported by Grants from The National Institute of Neurological Disorders and Stroke, National Institutes of Health. The Government of the United States of America may have certain rights in this invention.

### FIELD OF THE INVENTION

This invention is in the fields of neurology and pharmacology, and relates to drugs that can minimize brain injury due to various causes, such as traumatic head injury or crises such as stroke, cardiac arrest, or asphyxiation.

### BACKGROUND OF THE INVENTION

The fate of neurons undergoing hypoxic or ischemic injury is regulated by transcriptional and post-transcriptional events that contribute to competing cell-death and cell-survival programs (Sharp et al. (2000) J Cereb Blood Flow Metab 20: 1011-1032; Graham and Chen (2001) J Cereb Blood Flow Metab 21: 99-109). Survival-promoting events include the transcriptional induction or post-translational activation of neuroprotective proteins like erythropoietin (Sakanaka et al. (1998) Proc Natl Acad Sci USA 95: 4635-4640), vascular endothelial growth factor (Jin et al. (2000) Proc. Natl. Acad. Sci. U.S.A. 97: 10242-10247), and heme oxygenase (Dore et al. (1999) Mol Med 5: 656-663). In many cases, these are hypoxia-inducible proteins that help to counteract the adverse effects of hypoxia or ischemia by increasing anaerobic metabolism, tissue vascularity or oxygen delivery (Lopez-Barneo et al. (2001) Annu Rev Physiol 63: 259-287).

Another strategy for promoting the survival of metabolically active tissues like muscle or nerve may involve the tissue-specific expression of intracellular oxygen-binding proteins that can enhance oxygen extraction and intracellular diffusion, or neutralize reactive oxygen species. Examples include myoglobin in the case of muscle Suzuki and Imai (1998) Cell Mol Life Sci 54: 979-1004), and invertebrate nerve myoglobins (Dewilde et al. (1996) J Biol Chem 271: 19865-19870).

Neuroglobin (Ngb) is a newly discovered vertebrate globin that is expressed most abundantly in neurons (Burmester et al. (2000) Nature 407: 520-523). Ngb was identified by searching murine and human expressed sequence tag databases for partial globin-like sequences, then cloned and sequenced to reveal a 151-amino-acid protein with a predicted Mᵣ of ˜17 kDa, which exists as a monomer. Human and murine Ngbs show 94% sequence identity at the amino acid level, but limited homology to other known globins. For example, there is <21% sequence identity with vertebrate myoglobins and <25% identity with vertebrate hemoglobins. The protein that most closely resembles Ngb (30% amino acid identity) is the intracellular nerve myoglobin of the polychaete annelid worm Aphrodite aculeata (Dewilde et al. (1996) J Biol Chem 271: 19865-19870).

### SUMMARY OF THE INVENTION

This invention pertains to the discovery that neuroglobin (Ngb) expression is increased by neuronal hypoxia in vitro and focal cerebral ischemia in vivo, and that neuronal survival after hypoxia is reduced by inhibiting Ngb expression, e.g. with an antisense oligodeoxynucleotide (ODN) and enhanced by Ngb overexpression. Both induction of Ngb and its protective effect show specificity for hypoxia over other stressors. We conclude that hypoxia-inducible Ngb expression helps promote neuronal survival from hypoxic-ischemic insults.

Because Ngb is an oxygen-binding heme protein that is expressed preferentially in cerebral neurons, we investigated its possible involvement in neuronal responses to hypoxia or ischemia. The results indicate that Ngb is induced by neuronal hypoxia and cerebral ischemia and protects neurons from hypoxia in vitro, suggesting that Ngb may have a role in sensing or responding to neuronal hypoxia. In view of these results, we believe that Ngb expression provides a good target to screen for therapeutic agents that afford neuronal protection during or after hypoxia and/or an ischemic event.

Moreover, neuroglobin or agents that upregulate neuroglobin expression and/or activity can be used to mitigate one or more symptoms resulting from neurological damage associated with hypoxia and/or another ischemic event.

Thus, in one embodiment, this invention provides a method of screening for an agent that promotes neuronal survival from hypoxic-ischemic insult. The method involves contacting a cell with a test agent, and detecting the expression or activity of neuroglobin (Ngb) where an increase in neuroglobin expression or activity, as compared to the expression or activity of neuroglobin in a control indicates that the test agent is an agent promotes neuronal survival during or after hypoxic ischemic insult. In certain preferred embodiments, the cell is a neural cell and/or a neural tissue (e.g. a brain slice). The control can be a negative control (e.g. a cell contacted with said the agent at a lower concentration or a cell that is contacted with no test agent) or a positive control (e.g. a cell contacted with the test agent at a higher concentration that the test cell). In certain embodiments, the expression of neuroglobin (Ngb) is detected by detecting an Ngb nucleic acid (e.g. Ngb mRNA) from said cell (e.g. in a biological sample comprising said cell). In certain embodiments, the level of Ngb nucleic acid (e.g. Ngb mRNA) is measured by hybridizing the nucleic acid to a nucleic acid probe that specifically hybridizes to an Ngb nucleic acid (e.g. a probe that is complementary to all or to a part of an Ngb nucleic acid). Preferred probes are at least about 8 nucleotides in length, preferably at least about 10, 12, or 15 nucleotides in length, more preferably at least about 20, 25, or 30 nucleotides in length, and most preferably at least about 40, or 50 nucleotides in length. The hybridizing can be by any of a number of methods, e.g. a Northern blot, a Southern blot using DNA derived from an Ngb RNA, an array hybridization, an affinity chromatography, and an in situ hybridization. In certain embodiments, the Ngb probe is a member of a plurality of probes that forms an array of probes. In certain embodiments, the level of Ngb mRNA is measured using a nucleic acid amplification reaction (e.g. PCR, LGC, etc.).

The amount of Ngb gene product can also be detected by detecting the level of a neuroglobin (Ngb) protein from the cell (e.g. in a biological sample comprising the cell). Ngb protein can be detected by any of a number of methods known to those of skill in the art including, but not limited to capillary electrophoresis, a Western blot, mass spectroscopy, ELISA, immunochromatography, and immunohistochemistry. In certain embodiments, the cell (the test cell) is a cell cultured ex vivo. In certain embodiments, the test agent is administered to a mammal comprising a cell containing an Ngb nucleic acid or an Ngb protein. In certain embodiments, the test agent is administered to a brain section in culture.

It was also a discovery of this invention that hemin induces Ngb mRNA expression. Thus, in certain embodiments, hemin expression and/or activity can be used as a surrogate target to screen for agents that modulate neuroglobin expression and/or activity. Thus, rather than detecting changes in neuroglobin nucleic acid and/or proteins expression test agents can be screened for their ability to upregulate or downregulate hemin expression and/or activity.

In another embodiment, this invention provides a method of screening for an agent that promotes neuronal survival from hypoxic-ischemic insult. The method comprises providing a cell comprising an neuroglobin promoter and a reporter gene operably linked to the promoter, contacting the cell with a test agent, and detecting the expression or activity of the reporter gene where an increase in reporter gene, as compared to the expression or activity of the reporter gene in a control indicates that the test agent is an agent promotes neuronal survival during or after hypoxic ischemic insult. Preferred reporter genes include, but are not limited to chloramphenicol acetyl transferase (CAT), luciferase, β-galactosidase (β-gal), alkaline phosphatase, horse radish peroxidase (HRP), growth hormone (GH), and a fluorescent protein (e.g., green fluorescent protein (GFP), red fluorescent protein (RFP), etc.). The controls can include positive and/or negative controls, e.g. as described above.

In still another embodiment, this invention provides a method of prescreening for an agent that promoting neuronal survival from hypoxic-ischemic insult. The method involves contacting an Ngb nucleic acid or an Ngb protein with a test agent; and ii) detecting specific binding of the test agent to the Ngb nucleic acid or protein. The method can further involve recording test agents that specifically bind to said Ngb nucleic acid or protein in a database of candidate agents that promoting neuronal survival from hypoxic-ischemic insult. In certain embodiments, the test agent is not an antibody and/or not a protein, and/or not a nucleic acid. Preferred test agents include, but are not limited to small organic molecules. The detecting can comprise detecting specific binding of the test agent to an Ngb nucleic acid (e.g. via Northern blot, a Southern blot using DNA derived from a Ngb RNA, an array hybridization, an affinity chromatography, and an in situ hybridization). In certain embodiments, the detecting comprises detecting specific binding of the test agent to an Ngb protein (e.g. via capillary electrophoresis, a Western blot, mass spectroscopy, ELISA, immunochromatography, and immunohistochemistry). The test agent(s) can be contacted directly to the Ngb nucleic acid or to the Ngb protein, and/or contacted to a cell containing the Ngb nucleic acid and/or the Ngb protein, and/or contacted to an animal comprising a cell containing the Ngb nucleic acid or the Ngb protein. In certain embodiments, the cell is a cell (e.g. a neural cell) cultured ex vivo.

In still another embodiment, this invention provides a method of identifying a predilection to neural damage during a hypoxic or ischemic event in a mammal. The method involves obtaining a biological sample from the mammal; and detecting a mutation (e.g., an insertion, a deletion, a missense point mutation, and a nonsense point mutation) in an Ngb gene or gene product from the biological sample, where the presence of the mutation indicates a predilection to neural damage resulting from hypoxia or an ischemic event. The detection can be by any convenient method including, but not limited to a Southern blot, a DNA amplification, comparative genomic hybridization, immunohistochemistry, and cytogenetics. In certain embodiments, the detecting comprises detecting a mutation in a neuroglobin polypeptide (e.g. via capillary electrophoresis, a Western blot, mass spectroscopy, ELISA, immunochromatography, immunohistochemistry, etc.). In certain embodiments, the detecting comprises detecting a mutation in a neuroglobin nucleic acid (e.g. via hybridization techniques, amplification techniques, mass spectroscopy, and the like).

In another embodiment this invention provides a method of promoting neuronal survival from hypoxic ischemic insult. The method involves method comprising modulating the concentration and/or activity of an Ngb gene product in a neural cell of an organism. In certain embodiments, the method involves upregulating or repressing expression of a heterologous and/or an endogenous Ngb nucleic acid. In certain embodiments, the method involves upregulating or repressing expression of a heterologous and/or an endogenous hemin nucleic acid. In certain embodiments, the method involves upregulating or repressing expression of a component of the sGC-PKG pathway. The method can involve transfecting the cell with a vector that expresses (e.g. inducibly or constitutively) an Ngb protein and/or a hemin protein.

Also provided is a method of mitigating neurological damage associated with ischemia in a mammal where the method involves increasing hemin levels or upregulating hemin expression in the mammal.

This invention provides a method of modulating expression of neuroglobin where the method involves expression or activity of one or more components of the sGC-PKG pathway.

Definitions

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide. Preferred “peptides”, “polypeptides”, and “proteins” are chains of amino acids whose α carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free a-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to peptide mimetics such as amino acids joined by an ether as opposed to an amide bond.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The term “Ngb nucleic acid” refers to a nucleic acid that encodes a neuroglobin or the complement thereof. An “Ngb” nucleic acid can also include a fragment of a full length Ngb nucleic acid (preferably at least about 8 nucleotides in length, preferably at least about 10, 12, or 15 nucleotides in length, more preferably at least about 20, 25, or 30 nucleotides in length, and most preferably at least about 40, or 50 nucleotides in length.).

The term “reporter gene” refers to gene or cDNA that expresses a product that is detectable by spectroscopic, photochemical, biochemical, immunochemical, electrical, optical or chemical means. Useful labels in this regard include, but are not limited to fluorescent proteins (e.g. green fluorescent protein (GFP), red fluorescent protein (RFP), etc.), enzymes (e.g., horse radish peroxidase, alkaline phosphatase, β-galactosidase, and others commonly used in an ELISA), and the like

The term “reporter gene operably linked to a promoter” refers to a promoter and a reporter gene disposed such that the promoter regulates transcription of the reporter gene.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

The term “mammal” is used in accordance with standard usage. Thus, mammals include humans and non-human primates, as well as other mammals including, but not limited to canines, equines, porcines, felines, largomorphs, ungulates, bovines, rodents, murines, and the like.

The term “Ngb gene product” refers to a nucleic acid and/or a protein derived from an ngb nucleic acid (e.g. transcript such as ngb mRNA, a protein such as neuroblobin, fragments thereof, and the like).

As used herein, an “antibody” refers to a protein or glycoprotein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for. antigen recognition. The terms variable light chain (VL) and variable heavy chain (VH) refer to these light and heavy chains respectively.

Antibodies exist as intact immunoglobulins or as a number of well characterized fragments produced by digestion with various peptidases. Thus, for example, pepsin digests an antibody below (i.e. toward the Fc domain) the disulfide linkages in the hinge region to produce F(ab)′2, a dimer of Fab which itself is a light chain joined to V_{H}-C_{H}1 by a disulfide bond. The F(ab)′2 may be reduced under mild conditions to break the disulfide linkage in the hinge region thereby converting the (Fab′)2 dimer into an Fab′ monomer. The Fab′ monomer is essentially a Fab with part of the hinge region (see, Paul (1993) Fundamental Immunology, Raven Press, N.Y. for a more detailed description of other antibody fragments). While various antibody fragments are defined in terms of the digestion of an intact antibody, one of skill will appreciate that such fragments may be synthesized de novo either chemically, by utilizing recombinant DNA methodology, or by “phage display” methods (see, e.g., Vaughan et al. (1996) Nature Biotechnology, 14(3): 309-314, and PCT/US96/10287). Preferred antibodies include single chain antibodies, e.g., single chain Fv (scFv) antibodies in which a variable heavy and a variable light chain are joined together (directly or through a peptide linker) to form a continuous polypeptide.

The term “hypoxic ischemic insult” refers to neurological cell or tissue damage associated and/or pathological symptoms typically associated with neurological cell or tissue damage produced by reduced oxygen availability to the subject tissue (ie. partial or complete hypoxia). Sources of such ischemic insult/injury include, but are not limited to ischemia caused by myocardial infarction, stroke induced neuron death, reperfusion injury, traumatic head injury, cardiac arrest, asphyxiation, and the like.

The term “specifically binds”, as used herein, when referring to a biomolecule (e.g., protein, nucleic acid, antibody, etc.), refers to a binding reaction which is determinative of the presence biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent conditions” refers to conditions under which a probe will hybridize preferentially to its target subsequence, and to a lesser extent to, or not at all to, other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, chapt 2, Overview of principles of hybridization and the strategy of nucleic acid probe assays, Elsevier, N.Y. (Tijssen ). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook (1989) Molecular Cloning: A Laboratory Manual (2nd ed.) Vol. 1-3, Cold Spring Harbor Laboratory, Cold Spring Harbor Press, N.Y., and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

### BRIEF DESCRIPTION OF THE DRAWINGS

FIGS. 1A, 1B, 1C, and 1D: Neuronal hypoxia and ischemia induce Ngb protein expression. FIG. 1A: Representative Western blot showing increased Ngb expression in cultured cortical neurons maintained without oxygen for the indicated number of hours (left). The panel immediately beneath the Western blot shows Ngb MRNA expression over the same time course. Expression of the 17-kilodalton band (arrow) was quantified by computer densitometry (mean±SEM, n=3; *, P<0.05 relative to 0 h by t-test) (right). FIG. 1B: Representative Western blots (n=3) showing increased Ngb expression in cultures treated for 24 h with 300 μM Co2+ or 100 μM Dfx (left), but no change with 0.1 μM staurosporine (Stauro) or 500 μM SNP (right). FIG. 1C: Fluorescence labeling of cultured cortical neurons showing Ngb immunoreactivity in the cytoplasm of cells that express the neuronal nuclear antigen NeuN (left panel). Center panel shows the segregation of Ngb expression and DNA damage (detected by labeling with the Klenow fragment of DNA polymerase I, red) into distinct populations, corresponding to viable cells with large nuclei (DAPI staining, blue) and non-viable cells with shrunken nuclei. Preabsorption of the antibody with authentic Ngb peptide antigen abolished immunolabeling (right). FIG. 1D: Representative sections from contralateral, nonischemic rat cerebral cortex (left) and from penumbra (center) or core (right) of ischemic cerebral cortex at 24 h. Immunostaining for Ngb, shows increased Ngb expression in the penumbra; this increased staining is localized to the cytoplasm of normal-appearing, unshrunken cells with neuronal morphology (insets). Brown, anti-Ngb; blue, cresyl violet. Original magnification, ×400 (c and insets to d), ×200 (d).

FIGS. 2A, 2B, and 2C: Decreased Ngb expression exacerbates hypoxic neuronal death. FIG. 2A: Representative Western blot showing decreased Ngb expression compared to untransfected control cells (Con) in cultured cortical neurons treated with an antisense ODN directed against Ngb, but not with a sense ODN (left). Ngb expression was quantified (mean±SEM, n=3) by computer densitometry (*, P<0.05 relative to Con by t-test) (right). FIG. 2B: Cell viability, measured by MTT absorbance or trypan blue exclusion (TBE), in cultures maintained for 12 h without oxygen or in the presence of 0.1 μM staurosporine (Stauro) or 200-400 μM SNP, under standard conditions (no ODN) or after treatment with 5 μM sense or antisense ODN, added 3 h prior to the onset of and present throughout the toxic exposure (n=3-6). *, P<0.05 relative to no treatment (t-test). FIG. 2C: Fluorescence labeling of cultured cortical neurons treated with Ngb antisense (top panels) or sense (bottom panels) ODNs, showing immunoreactivity for the 17-20 kDa caspase-3 cleavage product (left panels, red), ODN fluorescence (center panels, green) and the merged images (right panels, yellow). Original magnification, ×400. Antisense-transfected cultures show an increase in the proportion of neurons that exhibit caspase-3 cleavage compared to sense-transfected cultures, consistent with the antisense-mediated decrease in cell viability shown in 2B.

FIGS. 3A, 3B, 3C, and 3D: Overexpression of Ngb reduces hypoxic cell death. FIG. 3A: Representative Western blot showing increased Ngb expression in cultured HN33 cells maintained without oxygen for the indicated number of hours (left). Expression of the 17-kilodalton band (arrow) was quantified by computer densitometry (mean±SEM, n=3; *, P<0.05 relative to 0 h by t-test) (right). FIG. 3B: pcDNA vector or Ngb-expressing recombinant plasmid (pcDNA-Ngb) was stably transfected into HN33 cells and overexpression of Ngb protein in pcDNA-Ngb-transfected cultures was confirmed by Western blotting (left). Ngb expression was quantified by computer densitometry (mean±SEM, n=5; *, P<0.05 relative to Con by t-test) (right). FIG. 3C: Cell viability, measured by MTT absorbance or trypan blue exclusion (TBE), in pcDNA- or pcDNA-Ngb-transfected cultures maintained for 8 h (Hyp8) or 24 h (Hyp24) without oxygen, or for 24 h in the presence of 0.1 μM staurosporine (Stauro) or 300 μM SNP. *, P<0.05 relative to untransfected control cultures (t-test). d, Oxygen consumption in untransfected (control) and pcDNA- or pcDNA-Ngb-transfected HN33 cells (5×10⁶ cells in 1 ml of DMEM) measured using a Clark oxygen electrode (Hansatech). Each tracing is the average of 3 independent experiments. The slopes give oxygen consumption (nmol O/ml/min), and were not significantly different across conditions (control, 7.46±0.90; pcDNA, 9.24±1.39; pcDNA-Ngb, 11.87±2.31; P=0.18 by ANOVA, n=9).

FIGS. 4A, 4B, and 4C show that hemin induces Ngb mRNA expression. FIG. 4A: HN33 cells were treated with hemin at the indicated concentrations for 24 h and RT-PCR was used to detect Ngb mRNA expression (left), which was quantified by computer densitometry and normalized to the expression of b-actin (right). *P<0.05, **P<0.001 relative to 0 μM. FIG. 4B: HN33 cells were treated with 50 μM hemin for the indicated times and RT-PCR was used to detect Ngb mRNA expression (left), which was quantified by computer densitometry and normalized to the expression of b-actin (right). *P<0.05, **P<0.001 relative to 0 h. FIG. 4C: HN33 cells were treated with 50 μM hemin for the indicated times and Northern blotting was used to detect Ngb mRNA expression (left), which was quantified by computer densitometry and normalized to the expression of β-actin (right). Data are representative blots (left) or means±SEM (right) from 3 experiments. *P<0.001 relative to 0 h.

FIGS. 5A and 5B show that hemin (0-50 μM) does not alter HN33 cell viability. HN33 cells were treated with hemin for 24 hours at the indicated concentrations (FIG. 5A) or at 50 μM for the indicated times (FIG. 5B), and viability was measured with MTT. Results (means±SEM from 3 experiments) are expressed as a percentage of viability in untreated control cultures. Only treatment with 100 μM hemin produced a significant (P<0.05) difference in viability relative to control.

FIGS. 6A- and 6B show that hemin induces Ngb protein expression. HN33 cells were treated with 50 μM hemin for the indicated times (left, 0-24 h; right, 1-3 d; C=c ontrol, H=hemin) and Western blotting was used to detect Ngb protein expression (FIG. 3 newA), which was quantified by computer densitometry (FIG. 3 newB). Data are representative blots (top) or means±SEM (bottom) from 3 experiments. *P<0.05, **P<0.001 relative to 0 h (unfilled bar, left) or to cultures maintained for the same period without hemin (unfilled bars, right).

FIG. 7, panels A, B, C, and D show that protein kinase inhibitors modify hemin-induced Ngb expression. HN33 cells were treated for 1 h with protein kinase inhibitors at the indicated concentrations, and then with 50 μM hemin for 24 h. Western blotting was used to detect Ngb protein expression (Panel A), which was quantified by computer densitometry (Panel B). Quantitative RT-PCR was used to detect Ngb mRNA expression (Panel C), which was quantified by computer densitometry and normalized to the expression of β-actin (Panel D). Data are representative blots (Panels A, C) or means±SEM (Panels B, D) from 3 experiments. The abbreviations are: Con, control; KT, KT5823 (PKG inhibitor); LY, LY83583 (sGC inhibitor); GF, GF109203X (PKC inhibitor); PD, PD98059 (MEK inhibitor). *P<0.05 relative to both control (unfilled bar) and hemin, **P<0.001 relative to control (unfilled bar) but not hemin.

FIG. 8, panels A, B, C, and D show that 8-Br-cGMP stimulates Ngb expression. HN33 cells were treated with 8-Br-cGMP (10 μM) for 2, 6, 12, or 24 h and Western blotting was used to detect Ngb protein expression (Panel A), which was quantified by computer densitometry (Panel B). Quantitative RT-PCR was used to detect Ngb MRNA expression (Panel C), which was quantified by computer densitometry and normalized to the expression of β-actin (Panel D). Data are representative blots (Panels A,C) or means±SEM (Panels B,D) from 3 experiments. *P<0.05, **P<0.001 relative to 0 h (unfilled bar).

FIG. 9 shows that hemin increases cGMP levels in HN33 cells. Cells were treated for the indicated times with 50 μM hemin, in the absence and presence of KT5823 (8 μM) LY83583 (1 μM, and cGMP levels were measured as described in Methods. Data are means±SEM from 4 experiments. *P<0.05 relative to treatment for 2 h with hemin alone (second bar from left), **P<0.01 relative to control (unfilled bar).

FIGS. 10A and 10B show the effects of protein kinase inhibitors on hypoxia-induced Ngb expression. HN33 cells were treated with protein kinase inhibitors at the indicated concentrations for 1 hour, and then exposed to hypoxia (95% N2/5% CO2) for 20 hours. Western blotting was used to detect Ngb protein expression (FIG. 10A), which was quantified by computer densitometry (FIG. 10B). Data are representative blots (top) or means±SEM (bottom) from 3 experiments. *P<0.05 for drug treatment relative to hypoxia alone.

### DETAILED DESCRIPTION

This invention pertains to the discovery that neuroglobin (Ngb) is increased by neuronal hypoxia in vitro and focal cerebral ischemia in vivo, and that neuronal survival after hypoxia is reduced by inhibiting Ngb expression with an antisense oligodeoxynucleotide (ODN) and enhanced by Ngb overexpression. Both induction of Ngb and its protective effect show specificity for hypoxia over other stressors. We conclude that hypoxia-inducible Ngb expression helps promote neuronal survival from hypoxic-ischemic insults.

Neuroglobin thus provides a good target to screen for agents that mitigate harmful effects from hypoxic-/ischemic insult. Methods are provided for screening for agents that promote neuronal survival from hypoxic-ischemic insult (e.g., ischaemic injury such as caused by myocardial infarction, stroke induced neuron death, reperfusion injury, traumatic head injury, cardiac arrest, asphyxiation, and the like).

In addition, it was discovered that neuroglobin (Ngb) can also be induced by hemin, and that this occurs in a dose- and time-dependent manner, at both the mRNA and . protein levels. We demonstrated further that induction of Ngb expression by hemin appears to be mediated by the sGC-PKG pathway.

Thus hemin and components of the sGC-PKG pathway also provide good targets to screen for modulators of neuroglobin expression and/or activity and that such modulators can be of significant value in mitigating neurological damage and/or affording neuroprotection during or after a hypoxic/ischemic event.

Thus, in certain embodiments, this invention contemplates methods of screening for modulators of neuroglobin expression and/or activity. In certain embodiments, this invention contemplates methods of screening for modulators of neuroglobin expression and/or activity using as a surrogate marker hemin expression or activity and/or expression or activity of one or more components of the sGC-PKG pathway.

In certain embodiments, this invention contemplates methods of reducing neurological damage and/or affording neuroprotection and/or mitigating one or more symptoms associated with a hypoxic ischemic insult (e.g. ischemia caused by myocardial infarction, stroke induced neuron death, reperfusion injury, traumatic head injury, cardiac arrest, asphyxiation, and the like.). The methods involve upregulating neuroglobin and/or hemin expression and/or activity and/or administering exogenous neruoglobulin and/or transfecting cells in the mammal to express heterologous neuroglobin and/or hemin.

I. Screening for Modulators of Neuroglobin Expression.

In one aspect, this invention pertains to the discovery that neuroglobin expression, is associated with hypoxic and/or ischemic neurological events. In particular, it was a surprising discovery that neuroglobin upregulation is protects neurological tissue during and/or after such an event. Thus, modulators (e.g. upregulators or downregulators) of neuroglobin expression and/or activity are of considerable interest and especially upregulators of neuroglobin expression and/or activity are useful in mitigating one or more symptoms associated with hypoxic ischemic injury. These modulators that can be useful in a wide variety of contexts (e.g. in the treatment one or more of the conditions described above).

In certain embodiments, the methods involve contacting a cell (preferably a cell from a particular target tissue (e.g., a neurological cell or tissue) with a test agent and detecting a change in expression or activity of neuroglobin. An increase in expression or activity of neuroglobin expression and/or activity indicates that the test agent can be useful in treating many of the conditions described herein. A decrease in expression or activity indicates that the test agent can be useful in some therapeutic contexts and is useful in a wide variety of research contexts.

When screening for modulators, a positive assay result need not indicate that particular test agent is a good pharmaceutical. Rather a positive test result can simply indicate that the test agent can be used to modulate expression or activity of neuroglobin r and/or can also serve as a lead compound in the development of other modulators (e.g., agonists).

Using known activities, and/or nucleic acid sequences, and/or amino acid sequences of neuroglobin, expression level(s) and/or activity can readily be determined according to a number of different methods, e.g., as described below. In particular, expression levels of neuroglobin can be altered by changes in the copy number of the gene(s) encoding neuroglobin, and/or by changes in the transcription of the gene product (i.e. transcription of ngb mRNA), and/or by changes in translation of the gene product (i.e. translation of the protein), and/or by post-translational modification(s) (e.g. protein folding, glycosylation, etc.). Thus useful assays of this invention include assaying for copy number, level of transcribed mRNA, level of translated protein, activity of translated protein, etc. Examples of such approaches are described below and illustrated herein in the examples.

It is also noted that hemin induces neuroglobin expression. Thus, in the assays described herein, it is possible to utilize hemin expression or activity (e.g. expression of hemin mRNA, or hemin polypeptide) as a surrogate marker for the ability of a test agent to upregulate neuroglobin expression.

A) Nucleic-Acid Based Assays.

1) Target Molecules.

Changes in expression level(s) of neuroglobin can be detected by measuring changes in mRNA encoding such component(s) and/or a nucleic acid derived from the mRNA (e.g. reverse-transcribed cDNA, etc.). In order to measure the expression level it is desirable to provide a nucleic acid sample (e.g. from the test tissue or cells) for such analysis. In preferred embodiments the nucleic acid is found in or derived from a biological sample. The term “biological sample”, as used herein, refers to a sample obtained from an organism or from components (e.g., cells) of an organism, or from cells in culture. The sample may be of any biological tissue or fluid. Biological samples may also include organs or sections of tissues such as frozen sections taken for histological purposes. In preferred embodiments the biological samples comprise neurological cells or tissues or other cells and/or tissues in which neuroglobin may be expressed.

The nucleic acid (e.g., ngb mRNA, DNA derived from ngb mRNA, etc.) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method and polyA+ mRNA is isolated by oligo dT column chromatography or by using (dT)n magnetic beads (see, e.g., Sambrook et al., (1989) Molecular Cloning: A Laboratory Manual (2nd ed.), Vols. 1-3, Cold Spring Harbor Laboratory, or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see. e.g, Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level (and thereby expression) of neuroglobin in a sample, the nucleic acid sample is one in which the concentration of the mRNA transcript(s), or the concentration of the nucleic acids derived from the mRNA transcript(s), is proportional to the transcription level (and therefore expression level) of the gene(s) of interest. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript or large differences of changes in nucleic acid concentration is desired, no elaborate control or calibration is required.

In the simplest embodiment, the nucleic acid sample is the total mRNA or a total cDNA isolated and/or otherwise derived from a biological sample (e.g. a neurological cell or tissue). The nucleic acid may be isolated from the sample according to any of a number of methods well known to those of skill in the art as indicated above.

2) Hybridization-Based Assays.

Using the known sequences for neuroglobin (see, e.g., GenBank Accession No: AF422797 and Zhang et al. (2002) Biochem. Biophys. Res. Commun. 290(5): 1411-1419), detecting and/or quantifying the transcript can be routinely accomplished using nucleic acid hybridization techniques (see, e.g., Sambrook et al. supra). For example, one method for evaluating the presence, absence, or quantity of reverse-transcribed cDNA involves a “Southern Blot”. In a Southern Blot, the DNA (e.g., reverse-transcribed mRNA), typically fragmented and separated on an electrophoretic gel, is hybridized to a probe specific for subject nucleic acid(s) (or to a mutant thereof). Comparison of the intensity of the hybridization signal from the probe with a “control” probe (e.g. a probe for a “housekeeping gene) provides an estimate of the relative expression level of the target nucleic acid.

Alternatively, the mRNA of interest can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target neuroglobin mRNA. Appropriate controls (e.g. probes to housekeeping genes) provide a reference for evaluating relative expression level.

An alternative means for determining the expression level(s) of neuroglobin is in situ hybridization. In situ hybridization assays are well known (e.g., Angerer (1987) Meth. Enzymol, 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In some applications it is necessary to block the hybridization capacity of repetitive sequences. Thus, in some embodiments, tRNA, human genomic DNA, or Cot-1 DNA is used to block non- specific hybridization.

3) Amplification-Based Assays.

In another embodiment, amplification-based assays can be used to measure expression (transcription) level of neuroglobin. In such amplification-based assays, the target nucleic acid sequences (e.g. neuroglobin nucleic acids etc.) act as template(s) in amplification reaction(s) (e.g. Polymerase Chain Reaction (PCR) or reverse-transcription PCR (RT-PCR)). In a quantitative amplification, the amount of amplification product will be proportional to the amount of template in the original sample. Comparison to appropriate (e.g. tissue or cells unexposed to the test agent) controls provides a measure of the target transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One typical internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y. The known nucleic acid sequence(s) for neuroglobin are sufficient to enable one of skill to routinely select primers to amplify any portion of the gene.

4) Hybridization Formats and Optimization of Hybridization Conditions.

i) Array-Based Hybridization Formats.

In one embodiment, the methods of this invention can be utilized in array-based hybridization formats. Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high-density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high-density arrays is also described in U.S. Pat. Nos. 5,744,305, 5,800,992 and 5,445,934.

ii) Other Hybridization Formats.

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labelled probes or the like. Other labels include ligands that bind to labeled antibodies, fluorophores, chemi-luminescent agents, enzymes, and antibodies that can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

ii) Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.).

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

iv) Labeling and Detection of Nucleic Acids.

The probes used herein for detection of neuroglobin expression levels can be full length or less than the full length of the target nucleic acid. Shorter probes are empirically tested for specificity. Preferred probes are sufficiently long so as to specifically hybridize with the target nucleic acid(s) under stringent conditions. The preferred size range is from about 10, 15, or 20 bases to the length of the target nucleic acid, more preferably from about 30 bases to the length of the target nucleic acid, and most preferably from about 40 bases to the length of the target nucleic acid. The probes are typically labeled, with a detectable label, e.g., as described above.

B) Detection of Expressed Neuroglobin Protein.

A) Assay Formats.

In addition to, or in alternative to, the detection of neuroglobin nucleic acid, alterations in expression of neuroglobin can be detected and/or quantified by detecting and/or quantifying the amount and/or activity of translated neuroglobin polypeptide.

The expression of neuroglobin can be detected and quantified by any of a number of methods well known to those of skill in the art. These can include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one embodiment, the neuroglobin is detected/quantified in an electrophoretic protein separation (e.g., a 1- or 2-dimensional electrophoresis). Means of detecting proteins using electrophoretic techniques are well known to those of skill in the art (see generally, R. Scopes (1982) Protein Purfication, Springer-Verlag, N.Y.; Deutscher, (1990) Methods in Enzymology Vol. 182: Guide to Protein Purification, Academic Press, Inc., N.Y.).

In another embodiment, Western blot (immunoblot) analysis is used to detect and quantify the presence of neuroglobin in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target, e.g., neuroglobin polypeptide, and can be directly labeled or alternatively may be subsequently detected using labeled antibodies (e.g., labeled sheep anti-mouse antibodies) that specifically bind to a domain of the antibody.

In certain embodiments, the neuroglobin polypeptide is detected using an immunoassay. As used herein, an immunoassay is an assay that utilizes an antibody to specifically bind to the analyte (e.g., the neuroglobin polypeptide(s),. The immunoassay is thus characterized by detection of specific binding of a neuroglobin to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (e.g., neuroglobin). In certain embodiments, the capture agent is an anti-neuroglobin antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Typical immunoassays for detecting the target polypeptide(s), e.g., neuroglobin, are either competitive or noncompetitive. Noncompetitive immunoassays are assays in which the amount of captured analyte is directly measured. In one “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (e.g., neuroglobin) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one embodiment, the antibody is immobilized on a solid substrate. The amount of target polypeptide bound to the antibody may be determined either by measuring the amount of target polypeptide present in a polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind neuroglobin, either alone or in combination. In the case where the antibody that binds neuroglobin is not labeled, a different detectable marker, for example, an enzyme-labeled antibody capable of binding to the monoclonal antibody which binds the neuroglobin, may be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention may also be other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds neuroglobin is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents may also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein can be routinely produced as described below.

B) Antibodies to Neuroglobin.

Either polyclonal or monoclonal antibodies can be used in the immunoassays of the invention described herein. Polyclonal antibodies are typically raised by multiple injections (e.g. subcutaneous or intramuscular injections) of substantially pure polypeptides or antigenic polypeptides into a suitable non-human mammal. The antigenicity of the target peptides can be determined by conventional techniques to determine the magnitude of the antibody response of an animal that has been immunized with the peptide. Generally, the peptides that are used to raise antibodies for use in the methods of this invention should generally be those which induce production of high titers of antibody with relatively high affinity for target polypeptides, such as neuroglobin.

If desired, the immunizing peptide can be coupled to a carrier protein by conjugation using techniques that are well-known in the art. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g. a mouse or a rabbit).

The antibodies are then obtained from blood samples taken from the mammal. The techniques used to develop polyclonal antibodies are known in the art (see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies see, for example, Coligan, et al. (1991) Unit 9, Current Protocols in Immunology, Wiley Interscience).

In certain embodiments, however, the antibodies produced will be monoclonal antibodies (“mAb's”). For preparation of monoclonal antibodies, immunization of a mouse or rat is preferred. The term “antibody” as used in this invention includes intact molecules as well as fragments thereof, such as, Fab and F(ab′)^{2′}, and/or single-chain antibodies (e.g. scFv) which are capable of binding an epitopic determinant.

The general method used for production of hybridomas secreting mAbs is well known (Kohler and Milstein (1975) Nature, 256:495). Briefly, as described by Kohler and Milstein the technique comprised isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody which bound to cancer cell lines. Confirmation of specificity among mAb's can be accomplished using relatively routine screening techniques (such as the enzyme-linked immunosorbent assay, or “ELISA”) to determine the elementary reaction pattern of the mAb of interest.

Antibody fragments, e.g. single chain antibodies (scFv or others), can also be produced/selected using phage display technology. The ability to express antibody fragments on the surface of viruses that infect bacteria (bacteriophage or phage) makes it possible to isolate a single binding antibody fragment, e.g., from a library of greater than 10¹⁰ nonbinding clones. To express antibody fragments on the surface of phage (phage display), an antibody fragment gene is inserted into the gene encoding a phage surface protein (e.g., pIII) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al (1990) Nature, 348: 552-554). Depending on the affinity of the antibody fragment, enrichment factors of 20 fold-1,000,000 fold are obtained for a single round of affinity selection. By infecting bacteria with the eluted phage, however, more phage can be grown and subjected to another round of selection. In this way, an enrichment of 1000 fold in one round can become 1,000,000 fold in two rounds of selection (McCafferty et al. (1990) Nature, 348: 552-554). Thus even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment natural V_{H} and V_{L} repertoires present in human peripheral blood lymphocytes are were isolated from unimmunized donors by PCR. The V-gene repertoires were spliced together at random using PCR to create a scFv gene repertoire which is was cloned into a phage vector to create a library of 30 million phage antibodies (Id.). From this single “naive” phage antibody library, binding antibody fragments have been isolated against more than 17 different antigens, including haptens, polysaccharides and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1:M to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

It will also be recognized that antibodies can be prepared by any of a number of commercial services (e.g., Berkeley antibody laboratories, Bethyl Laboratories, Anawa, Eurogenetec, etc.).

C) Reporter Gene Assays.

In another embodiment, the assays of this invention (e.g. assays for modulators of hemin or neuroglobin expression or activity can be performed using reporter gene assays. In such assays, a cell is provided comprising a reporter gene operably linked to the neuroglobin reporter (for assaying neuroglobin expression directly) and/or the hemin promoter (e.g. for assaying hemin expression). The cell is contacted with one or more test agents and the activity of the reporter gene or genes is detected and/or quantified.

An increase in reporter gene expression or activity (e.g. as compared to one or more controls) indicates that the test agent increases neuroglobin (or hemin) expression, while a decrease in reporter gene expression or activity (e.g. as compared to one or more controls) indicates that the test agent decreases neuroglobin (or hemin) expression.

Methods of providing reporter genes operably linked to particular promoters are well known to those of skill in the art (see, e.g., U.S. Pat. Nos. 6,391,641, 6,280,940, 5,897,990, and the like.).

D) Assay Optimization.

The assays of this invention have immediate utility in screening for agents that modulate (e.g. upregulate or downregulate) neuroglobinneuroglobin expression or activity in a cell, tissue or organism. The assays of this invention can be optimized for use in particular contexts, depending, for example, on the source and/or nature of the biological sample and/or the particular test agents, and/or the analytic facilities available. Thus, for example, optimization can involve determining optimal conditions for binding assays, optimum sample processing conditions (e.g. preferred isolation conditions), antibody conditions that maximize signal to noise, protocols that improve throughput, etc. In addition, assay formats can be selected and/or optimized according to the availability of equipment and/or reagents. Thus, for example, where commercial antibodies or ELISA kits are available it may be desired to assay protein concentration.

Routine selection and optimization of assay formats is well known to those of ordinary skill in the art.

II. Pre-Screening for Agents that Modulate Neuroglobin Expression or Activity.

In certain embodiments it is desired to pre-screen test agents for the ability to interact with (e.g. specifically bind to) neuroglobin and/or to a nucleic acid that encodes neuroglobin. Specifically, binding test agents, by interacting with the neuroglobin nucleic acid and/or protein are likely to alter neuroglobin expression and/or activity. Thus, in some preferred embodiments, the test agent(s) are pre-screened for binding to neuroglobin nucleic acid or protein before performing the more complex assays described above.

The test agent can be contacted directly to the neuroglobin nucleic acid or polypeptide, contacted to a cell containing the neuroglobin nucleic acid and/or protein, and/or to a tissue comprising such cells (e.g. to a lung tissue), and/or contacted to an animal (e.g., a mammal).

Such pre-screening can readily be accomplished with simple binding assays. Means of assaying for specific binding or the binding affinity of a particular ligand for a nucleic acid and/or for a protein are well known to those of skill in the art. In preferred binding assays, the neuroglobin nucleic acid and/or polypeptide, is immobilized and exposed to a test agent (which can be labeled), or alternatively, the test agent(s) are immobilized and exposed to the neuroglobin polypeptide or nucleic acid (which can be labeled). The immobilized moiety is then washed to remove any unbound material and the bound test agent or bound neuroglobin nucleic acid or protein is detected (e.g. by detection of a label attached to the bound molecule). The amount of immobilized label is proportional to the degree of binding between the neuroglobin nucleic acid and/or protein and the test agent.

In certain embodiments, the detecting is via a method selected from the group consisting of capillary electrophoresis, a Western blot, mass spectroscopy, ELISA, immunochromatography, and immunohistochemistry.

III. Scoring the Assay(s).

The assays of this invention are scored according to standard methods well known to those of skill in the art. The assays of this invention are typically scored as positive where there is a difference between the activity seen with the test agent present or where the test agent has been previously applied, and the (usually negative) control. In certain embodiments, the change is a statistically significant change, e.g. as determined using any statistical test suited for the data set provided (e.g. t-test, analysis of variance (ANOVA), semiparametric techniques, non-parametric techniques (e.g. Wilcoxon Mann-Whitney Test, Wilcoxon Signed Ranks Test, Sign Test, Kruskal-Wallis Test, etc.). Preferably the statistically significant change is significant at least at the 85%, more preferably at least at the 90%, still more preferably at least at the 95%, and most preferably at least at the 98% or 99% confidence level). In certain embodiments, the change is at least a 10% change, preferably at least a 20% change, more preferably at least a 50% change and most preferably at least a 90% change.

IV. Agents for Screening: Combinatorial Libraries (e.g., Small Organic Molecules)

Virtually any agent can be screened according to the methods of this invention. Such agents include, but are not limited to nucleic acids, proteins, sugars, polysaccharides, glycoproteins, lipids, and small organic molecules. The term small organic molecules typically refers to molecules of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one embodiment, high throughput screening methods involve providing a library containing a large number of potential therapeutic compounds (candidate compounds). Such “combinatorial chemical libraries” are then screened in one or more assays, as described herein to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library such as a polypeptide (e.g., mutein) library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) J. Med. Chem. 37(9): 1233-1250, Gallop et al. (1994) J. Med. Chem. 37(10): 1385-1401).

Preparation of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242), random bio-oligomers (PCT Publication WO 92/00091), benzodiazepines (U.S. Pat. No. 5,288,514), diversomers such as hydantoins, benzodiazepines and dipeptides (Hobbs et al. (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include, but are not limited to, automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist and the Venture™ platform, an ultra-high-throughput synthesizer that can run between 576 and 9,600 simultaneous reactions from start to finish (see Advanced ChemTech, Inc. Louisville, Ky.)). Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

V. High Throughput Screening

Any of the assays described herein are amenable to high-throughput screening (HTS). Moreover, the cells utilized in the methods of this invention need not be contacted with a single test agent at a time. To the contrary, to facilitate high-throughput screening, a single cell may be contacted by at least two, preferably by at least 5, more preferably by at least 10, and most preferably by at least 20 test compounds. If the cell scores positive, it can be subsequently tested with a subset of the test agents until the agents having the activity are identified.

High throughput assays for hybridizaiton assays, immunoassays, and for various reporter gene products are well known to those of skill in the art. For example, multi-well fluorimeters are commercially available (e.g., from Perkin-Elmer).

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

VI. Modulator Databases.

In certain embodiments, the agents that score positively in the assays described herein (e.g. show an ability to upregulate the expression or activity of neuroglobin) can be entered into a database of putative and/or actual modulators (e.g. agonists/upregulators) of the neuroglobin expression and/or activity. The term database refers to a means for recording and retrieving information. In certain embodiments the database also provides means for sorting and/or searching the stored information. The database can comprise any convenient media including, but not limited to, paper systems, card systems, mechanical systems, electronic systems, optical systems, magnetic systems or combinations thereof. Typical databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

VII. Mitigating Symptoms Associated with Hypoxia and/or an Ischemic Event.

It was a surprising discovery that neuroglobin expression is increased following a hypoxic ischemic insult and that increased neuroglobin expression provides neurological protection against a hypoxic ischemic insult. Moreover it was also discovered that hemin upregulation induces neuroglobin expression via the soluble guanylate cyclase-protein kinase G (sGC-PKG) pathway described by Ikuta et al. (2001) Proc Natl Acad Sci USA, 98:1847-1852).

Thus, in certain embodiments, this invention contemplates mnitigating symptoms (e.g. neurological damage) associated with a hypoxic ischemic insult (e.g. ischemia caused by myocardial infarction, stroke induced neuron death, reperfusion injury, traumatic head injury, cardiac arrest, asphyxiation, and the like) by upregulating neuroglobin expression or activity and/or by upregulating hemin expression and/or activity and/or by activating or upregulating one or more components of the sGC-PKG pathway.

Endogenous neuroglobin expression or activity can be increased by administration of one or more of the agents identified according to the screening methods described herein, and/or by administering one or more agents that increase hemin expression and/or by administering one or more agents that activate or upregulate one or more components of the sGC-PKG pathway.

In addition, exogenous neuroglobin can be provided by administration of neuroglobin and/or by transforming one or more cells with a vector that inducibly or constitutively expresses a heterologous hemin and/or neuroglobin.

1) Pharmaceutical Administration.

In order to carry out certain methods described herein one or more agents that upregulate neuroglobin expression and/or activity are administered to an individual at risk for or suffering from hypoxic ischemic insult. Typically where such “injury” has occurred, the agent is administered within 1 month of the injury, preferably within one week of the injury, more preferably within 24 hours of the injury and most preferably within one, two, three, or four hours of the injury. While this invention is described generally with reference to human subjects, veterinary applications are contemplated within the scope of this invention.

Various agents (e.g. neuroglobin, neuroglobin mimetics, etc.) can be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

The neuroglobin agonists and other agents that upregulate neuroglobin expression and/or activity are useful for parenteral, topical, oral, or local administration, such as by aerosol or transdermally, for prophylactic and/or therapeutic treatment of coronary disease and/or rheumatoid arthritis. The pharmaceutical compositions can be administered in a variety of unit dosage forms depending upon the method of administration. Suitable unit dosage forms, include, but are not limited to powders, tablets, pills, capsules, lozenges, suppositories, etc.

The agents and/or formulations thereof are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s). The excipients are preferably sterile and generally free of undesirable matter. These compositions may be sterilized by conventional, well known sterilization techniques.

The concentration of active agent(s) in the formulation can vary widely, and will be selected primarily based on fluid volumes, viscosities, body weight and the like in accordance with the particular mode of administration selected and the patient's needs.

In therapeutic applications, the compositions of this invention are administered to a patient at risk for or suffering from a condition characterized by hypoxic ischemic insult to a neurological tissue (e.g. a patient having or at risk for a stroke, myocardial infarction, acute neurological injury, etc.) in an amount sufficient to cure or at least partially arrest the neurological damage and/or its symptoms. An amount adequate to accomplish this is defined as a “therapeutically effective dose.” Amounts effective for this use will depend upon the severity of the disease/injury and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

In certain preferred embodiments, the agents are administered orally (e.g. via a tablet) or as an injectable in accordance with standard methods well known to those of skill in the art. In other preferred embodiments, the agents can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

The foregoing formulations and administration methods are intended to be illustrative and not limiting. It will be appreciated that, using the teaching provided herein, other suitable formulations and modes of administration can be readily devised.

2) “Genetic” Delivery Methods.

As indicated above, neuroglobin and/or hemin can be delivered and transcribed and/or expressed in target cells (e.g. neural cells) using methods of gene therapy. Thus, in certain preferred embodiments, the nucleic acids encoding neuroglobin and/or hemin are cloned into gene therapy vectors that are competent to transfect cells (such as human or other mammalian cells) in vitro and/or in vivo.

Many approaches for introducing nucleic acids into cells in vivo, ex vivo and in vitro are known. These include lipid or liposome based gene delivery (WO 96/18372; WO 93/24640; Mannino and Gould-Fogerite (1988) Bio Techniques 6(7): 682-691; Rose U.S. Pat. No. 5,279,833; WO 91/06309; and Felgner et al. (1987) Proc. Natl. Acad. Sci. USA 84: 7413-7414) and replication-defective retroviral vectors harboring a therapeutic polynucleotide sequence as part of the retroviral genome (see, e.g., Miller et al. (1990) Mol. Cell. Biol. 10:4239 (1990); Kolberg (1992) J. NIH Res. 4: 43, and Cornetta et al. (1991) Hum. Gene Ther. 2: 215).

For a review of gene therapy procedures, see, e.g., Anderson, Science (1992) 256: 808-813; Nabel and Felgner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 1149-1154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Böhm (eds) Springer-Verlag, Heidelberg Germany; and Yu et al., (1994) Gene Therapy, 1:13-26.

Widely used retroviral vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like).

The vectors are optionally pseudotyped to extend the host range of the vector to cells which are not infected by the retrovirus corresponding to the vector. For example, the vesicular stomatitis virus envelope glycoprotein (VSV-G) has been used to construct VSV-G-pseudotyped HIV vectors which can infect hematopoietic stem cells (Naldini et al. (1996) Science 272:263, and Akkina et al. (1996) J Virol 70:2581).

Adeno-associated virus (AAV)-based vectors are also used to transduce cells with target nucleic acids, e.g., in the in vitro production of nucleic acids and peptides, and in in vivo and ex vivo gene therapy procedures. See, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Construction of recombinant AAV vectors are described in a number of publications, including Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996. Other suitable viral vectors include, but are not limited to, herpes virus, lentivirus, and vaccinia virus.

V. Kits.

In still another embodiment, this invention provides kits for practice of the methods described herein. In certain embodiments the kits comprise a nucleic acid that hybridizes to a neuroglobin or hemin nucleic acid and/or an antibody that specifically binds to a neuroglobin and/or to a hemin polypeptide. Certain kits can comprise a vector that encodes a neuroglobin polypeptide and/or a hemin polypeptide and/or a cell containing such a vector.

The kits can optionally include any reagents and/or apparatus to facilitate practice of the methods described herein. Such reagents include, but are not limited to buffers, instrumentation (e.g. bandpass filter), reagents for detecting a signal from a detectable label, transfection reagents, cell lines, vectors, and the like.

In addition, the kits can include instructional materials containing directions (i.e., protocols) for the practice of the methods of this invention. Preferred instructional materials provide protocols utilizing the kit contents to screen for agents that increase or decrease neuroglobin expression and/or activity and/or to screen for agents that increase or decrease hemin expression and/or activity, and/or to screen for agents that upregulate or downregulate activity of the sGC-PKG pathway. Certain instructional materials can teach the neuroprotective effect of neuroglobin.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

VIII. Prognostics/Diagnostics—Detecting a Predilection to Neural Damage During a Hypoxic or Ischemic Event.

In certain embodiments, this invention provides methods of identifying mammals having a predilection to neural damage during a hypoxic or ischemic event. Such individuals are considered to be at greater physiological risk from such events (e.g. stroke, myocardial infarction, etc.). The methods generally involve detecting the presence of an abnormal neuroglobin or ngb gene and/or detecting the presence of an abnormal hemin or hemin gene. This is accomplished by any of a number of methods known to those of skill in the art. Thus for example, such methods can involve providing a biological sample from a subject mammal, detecting a mutation in an Ngb gene or gene product from the biological sample, where the presence of the mutation indicates a predilection to neural damage resulting from hypoxia or an ischemic event. Such mutations include, but are not limited to an insertion, a deletion, a missense point mutation, and a nonsense point mutation.

Methods of detecting mutations are well known to those of skill in the art. Such methods include, but are not limited to a Southern blot, a DNA amplification, comparative genomic hybridization, immunohistochemistry, and cytogenetics for nucleic acid detection and capillary electrophoresis, a Western blot, mass spectroscopy, ELISA, immunochromatography, and immunohistochemistry for protein detection. Methods of identifying mutations, e.g. single nucleotide polymorphism can be found for example in U.S. Pat. Nos. 6,410,231, 6,340,566, 5,952,174, 5,679,524, and the like.

### EXAMPLES

The following examples are offered to illustrate, but not to limit the claimed invention.

### Example 1

### Neuroglobin is Upregulated by and Protects Neurons from Hypoxic-Ischemic Injury

Materials and Methods

Cortical Neuron Culture.

Neuronal cultures were prepared from the cerebral hemispheres of 16-day Charles River CD1 mouse embryos, seeded at 3×10⁵ cells per well on 24-well culture dishes precoated with poly-D-lysine, and grown in Eagle's minimal essential medium (GIBCO BRL) with 5% horse serum and 5% fetal bovine serum (Jin et al. (2000) Proc. Natl. Acad. Sci. U.S.A. 97: 10242-10247). Cultures were treated with 10 μM cytosine arabinoside on day 6 and used on day 11, when >95% of cells expressed the neuronal marker, microtubule-associated protein 2. To induce hypoxia, cultures were placed in a modular incubator chamber (Billups-Rothenberg) containing humidified 95% air/5% CO2 (control), or humidified 95% N2/5% CO2 (hypoxic), for 0-24 h at 37° C., and then returned to normoxic conditions for the remainder, if any, of 24 h (Koretz et al. (1994) Brain Research 643: 334-337). Both control and hypoxic cultures contained 30 mM glucose. Cell viability was assayed by incubating cultures with 5 mg/ml of 3-(4,5-dimethylthiazol-2-yl)-2,5-diphenyl tetrazolium bromide (MTT) at 37° C. for 2 h and measuring absorbance at 570 nm in solubilized cells using a Cytofluor Series 4000 multi-well plate-reader (PerSeptive Biosystems). In some experiments, results were confirmed by trypan blue exclusion.

Western Blotting

Cell lysates were prepared as described (Jin et al. (2000) Proc. Natl. Acad. Sci. U.S.A. 97: 10242-10247) and 100-μg protein samples were electrophoresed on 12% SDS-PAGE gels and transferred to polyvinyldifluoridine membranes. Membranes were incubated overnight at 4° C. with a rabbit polyclonal antibody against Ngb (1:2000), which was produced by immunizing with a synthetic peptide corresponding to amino-acids 35-50 (NH2-CLSSPEFLDHIRKVML-COOH, , SEQ ID NO:1) of mouse Ngb, and affinity-purified using a Sulfolink kit (Pierce). A horseradish peroxidase-conjugated anti-rabbit secondary antibody (Santa Cruz Biotechnology) and a chemiluminescence substrate system (NEN) were used to visualize the immunolabeled bands (Id.).

Cytochemistry.

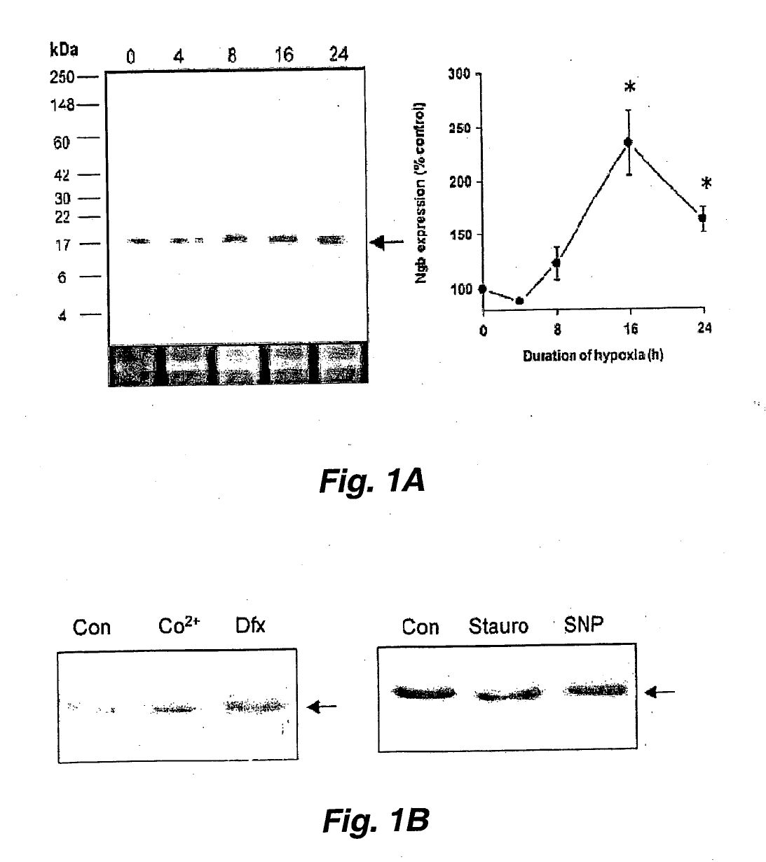

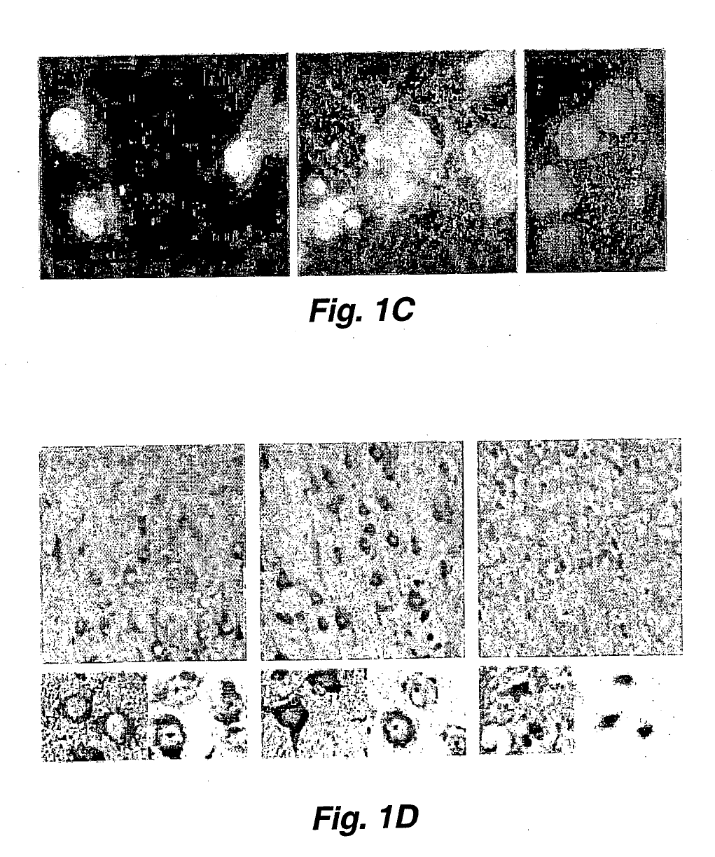

Cultures were fixed with 4% paraformaldehyde and incubated over night at 4° C. with one or more of the following primary antibodies: rabbit polyclonal anti-Ngb (1:200), mouse monoclonal anti-NeuN (1:200, Chemicon), and rabbit polyclonal anti-17-20 kDa caspase-3 cleavage product (1:100, New England Biolabs). The secondary antibodies (all 1:200) were fluorescein isothiocyanate-conjugated goat anti-rabbit or anti-mouse IgG (Vector) and rhodamine-conjugated donkey anti-rabbit IgG (Jackson ImmunoResearch). Controls for non-specific binding included omitting primary (FIG. 1C) or secondary antibodies. In addition, in some cultures, nuclei were labeled with 4′,6-diamidino-2-phenylindole (DAPI) or DNA strand breaks were labeled with the Klenow fragment of DNA polymerase I (Roche) followed by rhodamine avidin D (Vector), as described (Jin et al. (1999) J Neurochem 72: 1204-1214). Fluorescence signals were detected with a Nikon E800 epifluorescence microscope using excitation/emission wavelengths of 535/565 nm for rhodamine (red), 470/505 nm for fluorescein isothiocyanate (green) and 360/400 nm for DAPI (blue). Results were recorded with a Magnifire digital color camera (Optronics). To evaluate the in vivo expression of Ngb, immunohistochemistry was done on cerebral cortical sections from rats subjected to 90 min of focal cerebral ischemia followed by 4-24 h of reperfusion (Longa et al. (1989) Stroke 20: 84-91), using the same anti-Ngb primary antibody described above (1:200) and a horseradish peroxidase-conjugated goat anti-rabbit secondary antibody (1:1000, Santa Cruz Biotechnology).

ODN Treatment.

A phosphorothioate antisense ODN labeled with fluorescein at the 5′ end and directed against the initial coding region of the target Ngb mRNA (5′-TCC GGG CGC TCC AT-3′, from nucleotides 90-77, SEQ ID NO:2)was designed based on the mouse Ngb sequence obtained from Genbank (accession number NM 022414). This and a sense sequence (5′-ATG GAG CGC CCG GA-3′, SEQ ID NO:3), from nucleotides 77-90) were synthesized commercially (Operon Technologies) and purified by HPLC. Cultures were transfected with ODNs (1-10 μM) using FuGENE 6 (Roche), beginning 3 h before the onset of hypoxia. Cultures were analyzed by Western blotting and by MTT cell viability assay, and fluorescence microscopy was used to confirm transfection and examine its relationship to cell death, using caspase-3 activation as a marker.

HN33 Cell Culture and Transfection.

HN33 cells (passage number ≦20) were plated at 1×10⁵ cells/well on uncoated, 24-well plastic dishes and maintained as described (Shi et aL (1998) J Neurochem 70: 1035-1044.; Jin et al. (2000) J. Molec. Neurosci. 14: 197-203). Full-length mouse Ngb cDNA (Burmester et al. (2000) Nature 407: 520-523) was cloned into a pcDNA 3.1 plasmid with CMV promoter (Clontech). The recombinant plasmid (pcDNA-Ngb) or vector alone (pcDNA) was transfected into HN33 cells for 48 h using FuGENE 6 (Roche), followed by screening with G418 (Life Technology). Overexpression of Ngb was confirmed by Western blot as described above.

Results and Discussion

Hypoxia Induces Ngb Expression.

To test whether Ngb expression is induced in hypoxic cerebral cortical neurons, we generated and affinity-purified a rabbit polyclonal antibody against a synthetic peptide corresponding to amino acids 35-50 of mouse Ngb. This antibody labeled a band on Western blots prepared from mouse cortical neuron cultures at the predicted relative molecular mass of 17,000 (FIG. 1A). When cultures were deprived of oxygen for up to 24 h, expression of this protein increased, as did the abundance of Ngb mRNA, consistent with transcriptional induction. Expression was also increased by 300 μM CoCl2 and by 100 μM deferoxamine (Dfx) (FIG. 1B), which enhance the expression of hypoxia-inducible genes, including the major hypoxia-signaling transcription factor, hypoxia-inducible factor-1α (HIF-1α) (Semenza and Wang (1992) Mol. Cell. Biol. 12: 5447-5454). In contrast to the effects of hypoxia, CoCl2 and Dfx, other stressors—including staurosporine and the nitric oxide donor, sodium nitroprusside (SNP)—did not increase Ngb expression (FIG. 1B), suggesting the specific involvement of hypoxia-signaling pathways in Ngb induction.

Immunocytochemistry with the same anti-Ngb antibody used for Western blotting showed that Ngb was localized to the cytoplasm of cells that expressed the neuronal nuclear antigen NeuN, and were therefore neurons (FIG. 1C). In hypoxic cultures stained for Ngb and for DNA damage with the Klenow fragment of DNA polymerase I (Jin et aL (1999) J Neurochem 72: 1204-1214), Ngb was expressed most prominently in undamaged (Klenow-negative) cells. Ngb immunostaining was abolished when the antibody was preabsorbed with authentic Ngb peptide antigen.

To determine if Ngb expression was also increased by cerebral ischemia in vivo, we immunostained sections from cerebral cortex of mice subjected to focal cerebral ischemia by occlusion of the middle cerebral artery for 90 min followed by reperfusion for 4-24 h as described (Longa et al. (1989) Stroke 20: 84-91). These sections showed increased Ngb immunoreactivity in the cytoplasm of neurons from the ischemic compared to the nonischemic hemisphere (FIG. 1D). This increase was greatest in the ischemic penumbra and less pronounced in what would evolve into the ischemic core. These findings demonstrate that Ngb is expressed in neurons and that its expression is increased by hypoxia and ischemia, especially in neuronal populations that are destined to survive.

Reducing Neuroglobin Expression Worsens Hypoxic Injury.

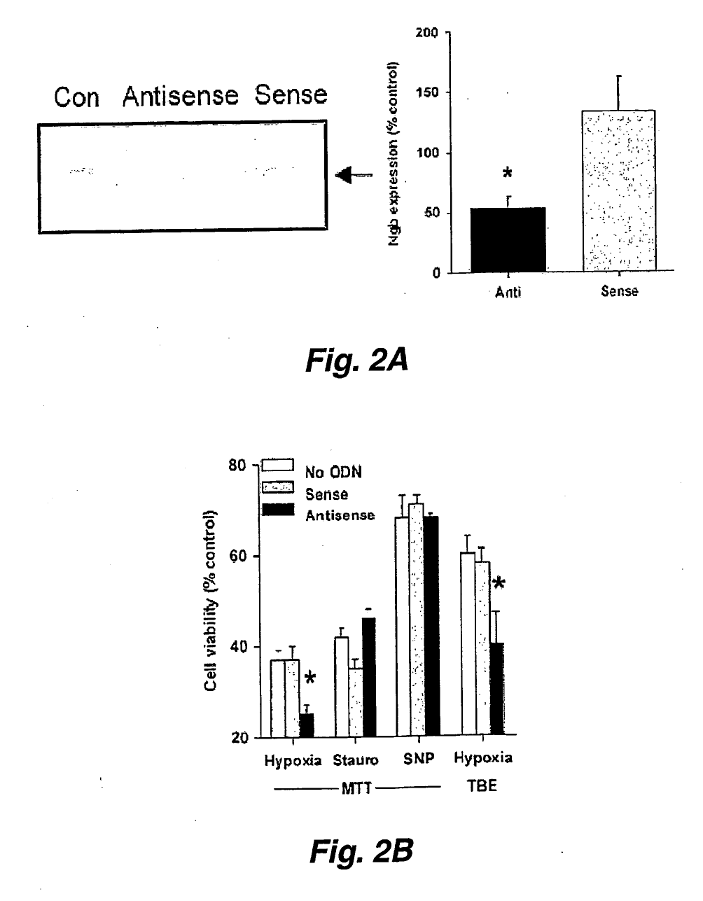

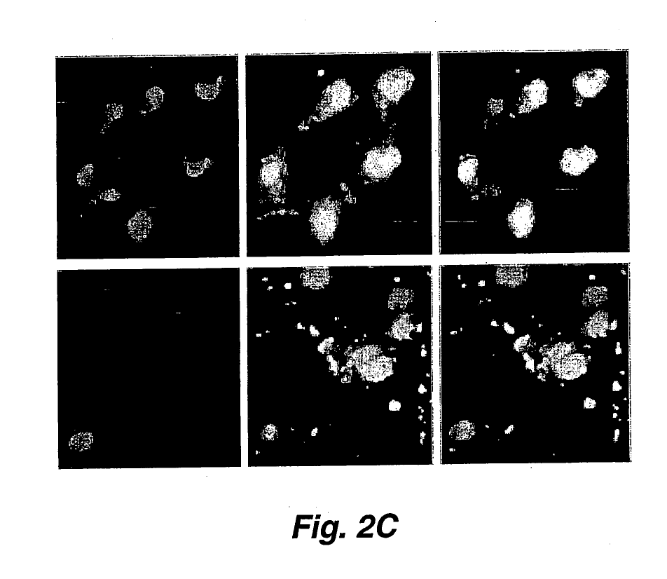

To begin to investigate the possibility that Ngb protects neurons from hypoxia, cultured neurons were transfected with a phosphorothioate antisense ODN directed against the initial coding region of the target Ngb mRNA (5′-TCC GGG CGC TCC AT-3′, from nucleotides 90-77, SEQ ID NO:4) or with a control sense sequence (5′-ATG GAG CGC CCG GA-3′, from nucleotides 77-90, SEQ ID NO:5), both labeled with fluorescein at the 5′ end. Transfection efficiency, measured in cultures transfected with the fluorescent Ngb antisense ODN and counterstained with DAPI, was 96±1% (n=10). Western blots showed that Ngb protein expression was reduced in antisense-transfected compared to sense-transfected or untransfected control cultures (FIG. 2A). The antisense-mediated reduction in Ngb expression was associated with a decrease in the viability of cultured neurons exposed to hypoxia, whether measured by MTT absorbance, which reflects mitochondrial function and is an early and sensitive indicator of cell injury in this model, or trypan blue exclusion, which relates to membrane integrity and declines with more advanced damage (FIG. 2B). In contrast to its effect in hypoxia, Ngb antisense had no effect on the toxicity of staurosporine or SNP. Fluorescence nicroscopy showed that many antisense-transfected cells, but few sense-transfected cells, co-expressed the 17-20 kDa caspase-3 cleavage product that is generated in neurons undergoing ischemic cell death (Namura et al. (1998) J Neurosci 18: 3659-3668) (FIG. 2C).

Increasing Neuroglobin Expression Lessens Hypoxic Injury.

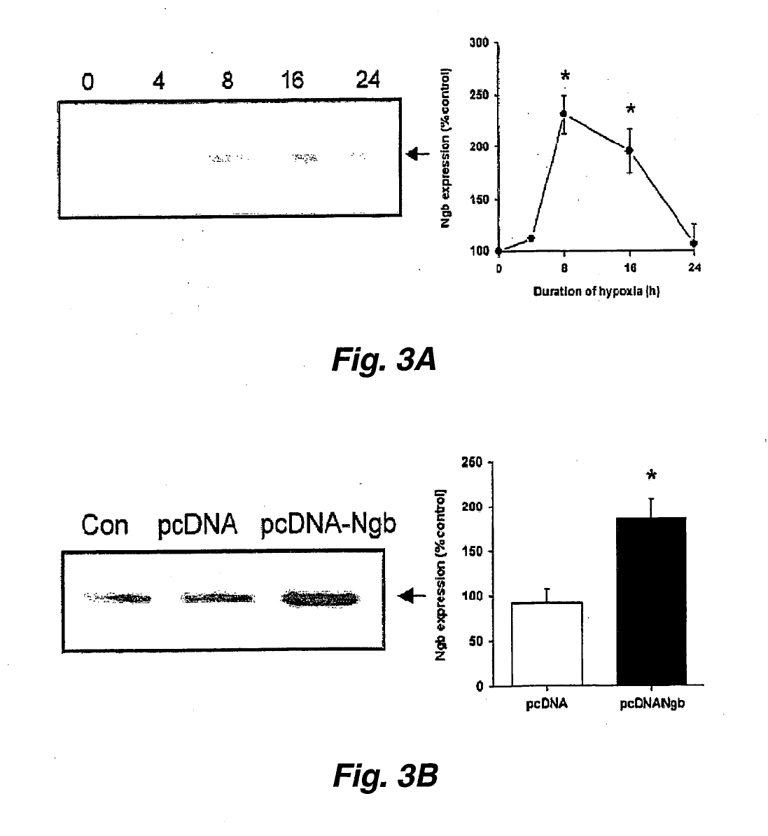

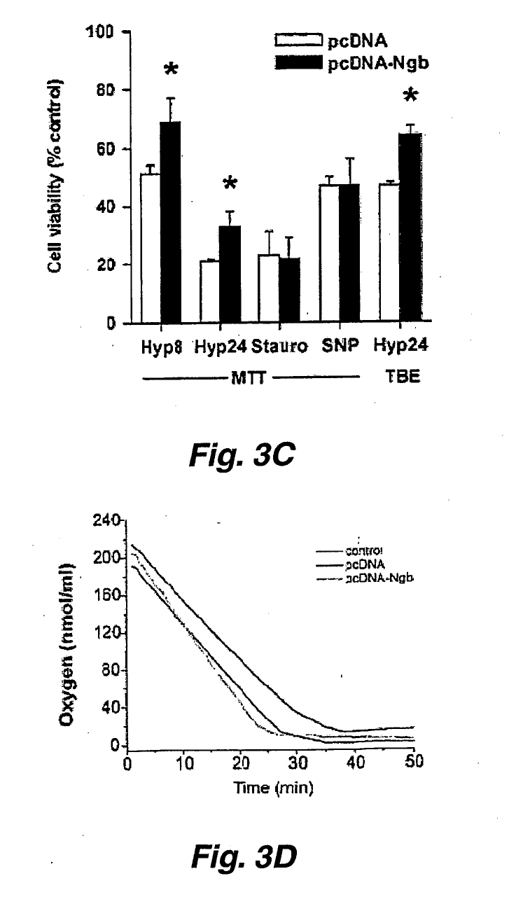

To test further the protective effect of Ngb in hypoxia, full-length mouse Ngb cDNA (Burmester et al. (2000) Nature 407: 520-523) was cloned into a pcDNA 3.1 plasmid with CMV promoter (pcDNA-Ngb) and transfected into and stably expressed in HN33, an immortalized hippocampal neuronal cell line (Lee et al. (1990) J Neurosci 10: 1779-1787). These cells were chosen because they provided high transfection efficiency, and because their response to hypoxia is well-characterized (Jin et al. (2000) Proc. Natl. Acad Sci. U.S.A. 97: 10242-10247; Shi et al. (1998) J Neurochem 70: 1035-1044). Hypoxia increased the expression of Ngb in HN33 cells, with a time course similar to that observed in cultured cortical neurons (FIG. 3A). Transfection with pcDNA-Ngb led to overexpression of Ngb (FIG. 3B) and increased the viability of hypoxic HN33 cells, determined with either MTT or trypan blue (FIG. 3C). However, pcDNA-Ngb afforded no protection against staurosporine or SNP.

Possible Mechanisms for Induction of Ngb.

These results are consistent with a role for Ngb as a hypoxia-inducible neuroprotective factor in hypoxic-ischemic injury. How hypoxia stimulates Ngb expression is uncertain, although hypoxia can induce hemoglobin synthesis in invertebrates (Weber and Vinogradov (2001) Physiol Rev 81: 569-628) and may act through HIF-1 to regulate β-globin gene expression during vertebrate development (Bichet et al. (1999) FASEB J 13: 285-295). The effects of CoCl2 and Dfx (FIG. 1B) are consistent with involvement of HIF-1 in hypoxic induction of Ngb expression, as is the observation that the 5′-untranslated region of Ngb (Genbank accession number NM 022414) contains several copies of the consensus HIF-1 binding sequence 5′-RCGTG-3′ (SEQ ID NO:6) (Semenza et al. (1996) J Biol Chem 271: 32529-32537), located 2073, 1977, 1445, 1041, 985, 627, 522 and 64 nucleotides upstream of the transcription initiation site.

Possible Mechanisms for Protection by Ngb.

The manner in which Ngb exerts its neuroprotective effect is also uncertain. One possibility is that, like myoglobin in muscle, it may bind oxygen and facilitate its delivery to mitochondria (Suzuki and Imai (1998) Cell Mol Life Sci 54: 979-1004). To evaluate this possibility, we used a Clark oxygen electrode (Minning et al. (1999) Nature 401: 497-502) to compare oxygen consumption in control and Ngb-overexpressing HN33 cells (FIG. 3D). Oxygen consumption did not vary significantly across conditions, indicating that Ngb does not increase the rate of oxygen consumption, and arguing for a different mode of neuroprotective action. In some respects, this is not surprising, because oxygen supply does not normally limit oxygen consumption, and because the affinity of Ngb for oxygen may be too high for it to release oxygen under physiological conditions (Trent et al. (2001) J Biol Chem 276: 30106-30110), although this is disputed (Couture et al. (2001) J Biol Chem 276(39): 36377-36382; Dewilde et al. (2001) J Biol Chem., 276(42): 38949-28955). Alternatively, and also by analogy to myoglobin, Ngb might scavenge nitric oxide (Flögel et al. (2001) Proc Natl Acad Sci USA 98: 735-740), which has been implicated in hypoxic-ischemic neuronal injury (Dawson et al. (1996) J Neurosci 16: 2479-2487). However, the failure of Ngb antisense to exacerbate and of Ngb overexpression to protect against SNP toxicity in our model argues against this mechanism. Additional possibilities are that Ngb might be involved in sensing hypoxia and triggering protective cellular responses thereto, or in detoxifying mediators of hypoxic-ischemic injury other than nitric oxide, for both of which actions there is precedent among nonvertebrate globins (Weber and Vinogradov (2001) Physiol Rev 81: 569-628).

Conclusion.

The recent discovery of Ngb (Burmester et al. (2000) Nature 407: 520-523) and the results presented here provide evidence for the existence of a novel endogenous neuroprotective mechanism. Understanding how Ngb and other hypoxia-inducible proteins confer neuronal protection will facilitate the development of improved treatment for ischemic disorders such as stroke.

### Example 2

### Hemin Induces Neuroglobin Expression in Neural Cells

Neuroglobin is a newly identified vertebrate globin that binds O₂ and is expressed in cerebral neurons. We found recently that neuronal expression of neuroglobin is stimulated by hypoxia and ischemia and protects neurons from hypoxic injury. Here we report that, like hemoglobin and myoglobin, neuroglobin expression can also be induced by hemin. Induction was concentration- and time-dependent, with maximal (about 4-fold) increases in neuroglobin MRNA and protein levels occurring with 50 μM hemin and at 8-24 hours. The inductive effect of hemin was attenuated by the protein kinase G inhibitor KT5823 and the soluble guanylate cyclase inhibitor LY83583, was mimicked by treatment with 8-Br-cGMP, and was accompanied by a >10-fold increase in cGMP levels, suggesting that it is mediated through protein kinase G and soluble guanylate cyclase. In contrast, hypoxic induction of neuroglobin was blocked by the mitogen-activated protein kinase/extracellular signal-regulated kinase kinase (MEK) inhibitor PD98059, indicating that hemin and hypoxia regulate neuroglobin expression by different mechanisms. These results provide evidence for regulation of neuroglobin expression by at least two signal transduction pathways.

Introduction

Globins are porphyrin-containing proteins known for their oxygen-carrying capacity. They are important in all organisms utilizing oxygen (Bunn and Poyton (1996) Physiol Rev. 76:839-885). Three types of globins have been described in vertebrates: hemoglobin, found in blood; myoglobin, located in muscle; and neuroglobin (Ngb), newly identified in the nervous system.² Although Ngb consists of single chains with 151 amino acids that share only 21-25% sequence identity with hemoglobin and myoglobin, it conserves the key amino acid residues that are required for hemoglobin and myoglobin function (Burmester et al. (2000) Nature, 407:520-523).

Ngb is a heme protein. It contains a proximal His residue that coordinates with heme, a distal His residue that may interact with heme-bound ligands, and a Phe residue involved in interactions with heme (Id.). Ngb has a moderate oxygen affinity, 2 torr, about 2-fold lower than that of myoglobin but higher than that of hemoglobin. It has been proposed that Ngb could have a function similar to that of myoglobin, and serve to transport oxygen to neuronal mitochondria (Id.). We reported recently that neuronal hypoxia and ischemia increase Ngb expression and that this may help to promote neuronal survival from hypoxic-ischemic insults, since survival is reduced by inhibiting Ngb expression and enhanced by Ngb Overexpression (Sun et al. (2001 Proc Natl Acad Sci USA. 98:15306-15311).

Heme is a prosthetic group in numerous enzymes, cytochromes and globins, which are involved in transport and storage of oxygen, generation of energy by respiration, and controlling oxidative damage. It plays key roles in oxygen sensing and utilization in virtually all organisms (Bunn and Poyton (1996) Physiol Rev. 76:839-885). Further, heme directly regulates numerous molecular and cellular processes in systems that sense or use oxygen (Lok and Ponka (1999) J Biol Chem. 1999;274:24147-24152; Badr et al. (1999) Brain Res Mol Brain Res, 64:24-33), including cell differentiation, transcription, translation, and protein translocation and assembly (Padmanaban et al. (1989) Trends Biochem Sci., 14:493-496; Sassa et al. (1996) Int J Hematol., 63:167-178; Zhu and Zhang (1999) Biochem Biophys Res Commun., 258:87-93).

Heme is also critical for erythropoiesis (Nakajima et al. (1999) EMBO J., 18:6282-6289). Hemin, the ferric chloride salt of heme, stimulates gene transcription, translation, and assembly of hemoglobin and other erythroid-specific proteins and enzymes (Rutherford et al. (1979) Nature, 280:164-165; Andersson et al. (1979) Int J Cancer, 24:514; Dean et al. (1983) Proc Natl Acad Sci USA., 80:5515-5519). Like hemoglobin, myoglobin can also be induced by hemin in a dose-dependent manner (Graber et al. J Biol Chem., 261:9150-9154) Induction of hemoglobin by hemin in K562 human erythroleukemia cells is reported to be mediated by extracellular signal-regulated kinase (Erk1/2) (Woessmann et al. (2001) Exp Cell Res., 264:193-200). Recently, induction of fetal globin gene expression by hemin in K562 cells has been found to be regulated by the soluble guanylate cyclase-protein kinase G (sGC-PKG) pathway (Ikuta et al. (2001) Proc Natl Acad Sci USA, 98:1847-1852). Protein kinase C (PKC) is also involved in hemin-induced gene expression and erythroid differentiation. Inhibition of PKC stimulates erythroid differentiation and hemoglobin expression in HEL cells (Yumoto et al. (1990) J Cell Physiol., 143:243-250; Hong et al. (1997) Blood, 87:123-131).

The structural and functional similarity of Ngb to hemoglobin and myoglobin suggests that Ngb may also be a hemin-responsive gene. Therefore, we treated HN33 cells, an immortalized cell line derived from somatic cell fusion of mouse hippocampal neurons and N18TG2 neuroblastoma cells (Lee et al. (1990) J Neurosci. 10:1779-1787; Jin et al. (2000) J Molec Neurosci. 14:197-203; Shi et al. (1998) J Neurochem., 70:1035-1044),with hemin and measured the expression of Ngb. Hemin induced Ngb expression at both the mRNA and protein levels. Blocking sGC-PKG activity inhibited the induction of Ngb expression by hemin, whereas a cGMP analog increased expression. These results suggest that Ngb is a hemin-responsive gene and that its expression is mediated by the sGC-PKG pathway.

Methods.

Chemicals

Hemin and 8-Br-cGMP were purchased from Sigma (St. Louis, Mo.). The PKG inhibitor KT5823, the sGC inhibitor LY83583, the mitogen-activated protein kinase/extracellular signal-regulated kinase kinase (MEK) inhibitor PD98059, and the protein kinase C (PKC) inhibitor GF109203X were from Calbiochem (San Diego, Calif.).

Cell Culture

HN33 cells were cultured as described (Lee et al. (1990) J Neurosci. 10: 1779-1787; Shi et al. (1998) J Neurochem., 70:1035-1044; Jin et al. (2000) Proc Nati Acad Sci USA., 97:10242-10247). Cells were plated at 4×10⁵ cells/well onto uncoated, 6-well plastic culture dishes in Dulbecco's modified Eagle's medium containing 10% (v/v) fetal bovine serum, 100 units/ml of penicillin and 100 μg/ml of streptomycin, and maintained at 37° C. in humidified 95% air/5% CO₂. Cells were treated with hemin (typically 50 μM) or 8-Br-cGMP (10 μM) for up to 3 days, without daily replacement. To suppress sGC or PKG activity, cells were pretreated for 1 hour with inhibitors before adding other reagents.

RT-PCR and Northern Blot Analyses

RT-PCR and Northern blot analyses were carried out as described previously (Zhu and Zhang (1999) Biochem Biophys Res Commun., 258:87-93). Briefly, total RNA from treated and untreated cells was extracted using a RNeasy Mini Kit (QIAGEN Operon, Valencia, Calif.), and DNA-free total RNA (2 μg per sample) was reverse-transcribed into first-strand cDNA using the Reverse Transcription System and Oligo-dT₁₂₋₁₈ (GIBCO-BRL, Rockville, Md.) (Id.). Sequences of the primers used for PCR amplification were: Ngb forward, 5′ CTC TGG AAC ATG GCA CTG TC 3′ (nt 135-154, SEQ ID NO:7); Ngb reverse, 5′ GCA CTG GCT CGT CTC TTA CT 3′ (nt 547-528, SEQ ID NO:8); β-actin forward, 5′ CAC AGG CAT TGT GAT GGA CTC 3′ (nt 524-544, SEQ ID NO:9); β-actin reverse, 5′ GCT CAG GAG GAG CAA TGA TCT 3′ (nt 582-563, (SEQ ID NO:10). Primers were designed based on the published sequences of these genes and were synth esized by QIAGEN Operon. All PCR primer pairs gave rise to only one discrete band of the expected size. PCR reactions were carried out in a total volume of 25 μl containing 2 μl cDNA, 1×PCR buffer (Boehringer Mannheim, Mannheim, FRG), 200 μM dNTPs, 0.6 U Taq DNA polymerase (Boehringer Mannheim), and 0.2 μM primers. The optimal conditions for amplification (temperature and cycle number) were determined experimentally according to previously published procedures (Id.). Different ratios of Ngb to b-actin primer pairs were tested to ensure that Ngb and b-actin were amplified with similar efficiency, and a kinetic study was undertaken to establish the number of cycles sufficient to detect both Ngb and b-actin without reaching saturation for either. The parameters chosen for PCR amplification were 95° C. for 1 minute, 57° C. for 45 seconds, 72° C. for 1 minute for 25-30 cycles, and a final incubation at 72° C. for 10 minutes. PCR products were separated on 1.2% agarose gels, visualized by ethidium bromide staining, and quantified using a ChemiImage System (Alpha Innotech Corporation, San Leandro, Calif.).

For Northern blotting, 15 μg of total RNA from each sample was fractionated on 1% formaldehyde/agarose gels and transferred to Hybond-N nylon membranes (Amersham Pharmacia, Piscataway, N.J.). Filters were hybridized with probes for Ngb MRNA, and β-actin mRNA at 68° C. in hybridization buffer (Clontech, Palo Alto, Calif.). ³²P-radiolabeled DNA probes were synthesized using cDNA obtained from RT-PCR amplification.

Ouantitative RT-PCR

Quantitative RT-PCR analysis was carried out as described previously.8 PCR reactions were carried out in a total volume of 25 μl containing 1 μI cDNA, 1×PCR buffer (Boehringer Mannheim), 200 μM dNTPs, 0.6 U Taq DNA polymerase (Boehringer Mannheim), two pairs of primers (one pair for the Ngb gene, another for the b-actin gene used as an internal control). The optimal conditions for amplification (the proportion between the two pairs of primers, temperatures, and cycle numbers) were determined experimentally according to previously established procedures.8 PCR products were separated on 1.2% agarose gels, visualized by ethidium bromide staining, and quantified using a ChemiImage System (Alpha Innotech Corporation).

Western Blotting

Cells were washed twice in PBS, and whole-cell extracts were prepared by adding 10 volumes of 1× sample buffer containing 2% SDS, 100 mM dithiothreitol, 60 mM Tris (pH 6.8) and 10% glycerol, and boiled for 5 minutes. Protein concentrations were determined using Bradford Protein Assays (Bio-Rad, Hercules, Calif.); 30 μg of protein was analyzed by 12% or 15% SDS-PAGE and transferred to Immuno-Blot PVDF membranes (Bio-Rad). Membranes were probed with affinity-purified anti-Ngb antibody, which was produced by immunizing with a synthetic peptide corresponding to amino-acids 35-50 (NH₂-CLSSPEFLDHIRKVML-COOH, (SEQ ID NO:11) of mouse Ngb (Sun et aL (2001 Proc Natl Acad Sci USA. 98:15306-15311),and the signal was detected with Boehringer Mannheim chemiluminescence blotting kits. Differences in protein expression on Western blots were quantified using a GS-710 calibrated imaging densitometer and Quantity One software (Bio-Rad).

Cell Viability Assays

Cell viability was assessed by measuring formazan produced by the reduction of 3-(4,5-dimethylthiazol-2-yl)-2,5-diphenyl tetrazolium bromide (MTT) in viable cells.3 Cells were incubated with 5 mg/ml of MTT (Sigma) at 37° C. for 2 hours. The medium was removed and cells were solubilized with dimethylsufoxide and transferred to 96-well plates. The formazan reduction product was detected by measuring absorbance at 570 nm in a Cytofluor Series 4000 multi-well plate-reader (PerSeptive Biosystems, Framingham, Mass.). Results were expressed as a percentage of control absorbance, measured in normoxic cultures, after subtracting background absorbance (measured in freeze-thawed cultures) from all values.

Measurement of Intracellular cGMP Content in HN33 Cells

HN33 cells (4×10⁴ cells in 100 μl) were plated on 96-well microtiter plates and treated with 50 μM hemin for 2, 8, 16, or 24 hours. Intracellular cGMP concentrations were measured in quadruplicate using a cGMP enzyme-immunoassay system (Amersham Pharmacia).

Data Analysis

Quantitative data were expressed as mean±SEM from at least 3 experiments. ANOVA and Student's t test were used for statistical analysis, with p<0.05 considered significant.

Results

Hemin Induces Ngb mRNA Expression

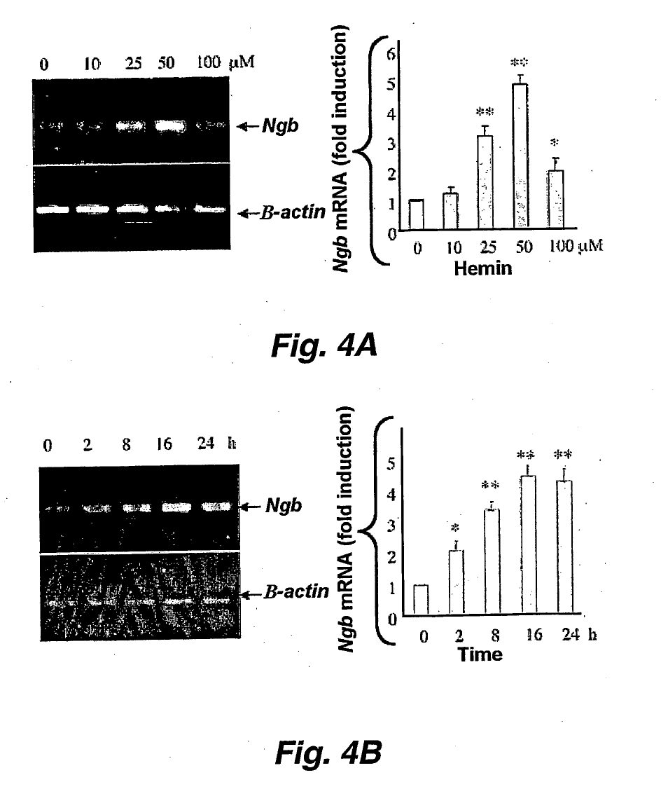

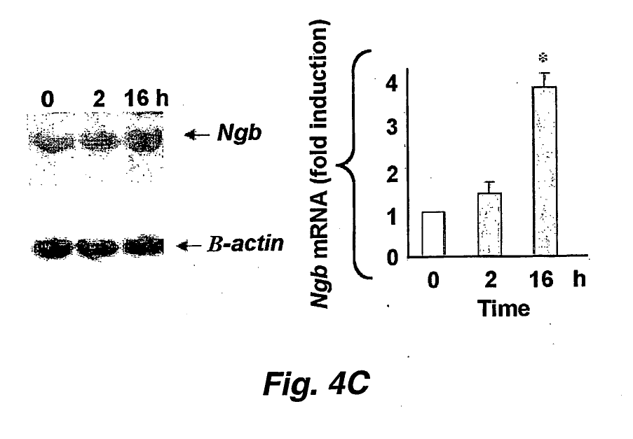

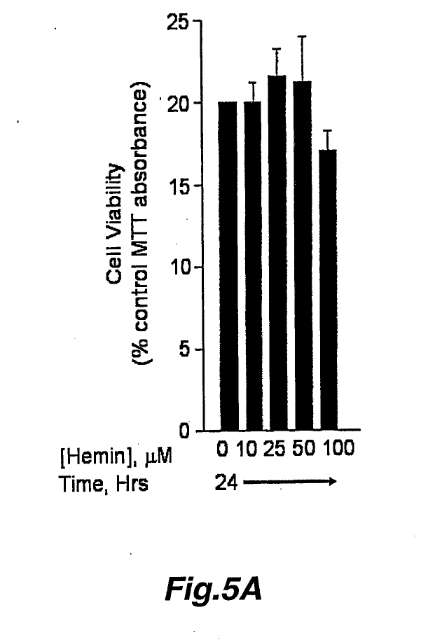

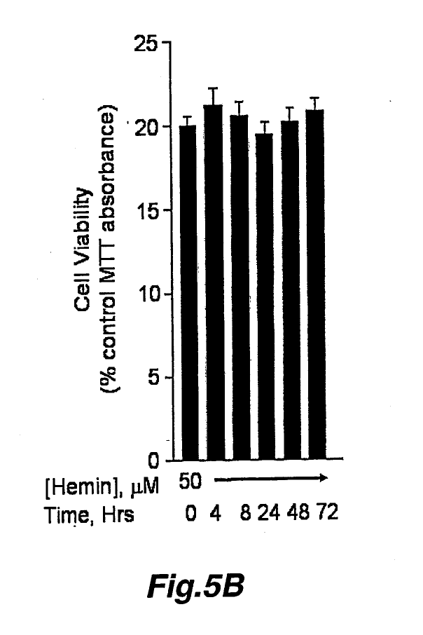

Hemin stimulates K562 erythroleukemia cells to synthesize erythroid-specific protein s, such as embryonic and fetal globins (Rutherford et al. (1979) Nature, 280:164-165; Andersson et al. (1979) Int J Cancer, 24:514). When K562 cells are treated with 50 μM hemin for 2-3 days, more than 50% of the cells produce a high level of hemoglobin (Zhu and Zhang (1999) Biochem Biophys Res Commun., 258:87-93; Partington and Patient (1999) Nucleic Acids Res., 27:1168-1175). To study if hemin induces Ngb expression in HN33 cells, we first measured Ngb expression at the mRNA level. Cells were treated with 10 to 100 μM hemin for 24 hours, and total RNA from these cells was isolated and reverse transcribed. RT-PCR analysis showed that hemin induced Ngb mRNA expression, normalized for β-actin mRNA expression, in a dose-dependent manner (FIG. 4A). Maximal (4-fold) induction occurred at 25-50 μM. To determine the time course of Ngb induction, HN33 cells were treated with 50 μM hemin for 2 to 24 hours. Ngb mRNA was induced in a time-dependent manner, with maximal induction between 8 and 24 hours (FIG. 4B). To verify these results, Ngb mRNA levels in HN33 cells were also measured by Northern blot analysis. Consistent with the results of RT-PCR analysis, Ngb mRNA levels were enhanced about 4-fold after treatment with 50 μM hemin for 16 hours (FIG. 4C). Treatment with 50 μM hernin for up to 3 days had no effect on cell viability, although 100 μM hemin reduced viability by ˜15% (FIG. 5).

Hemin Induces Ngb Protein Expression

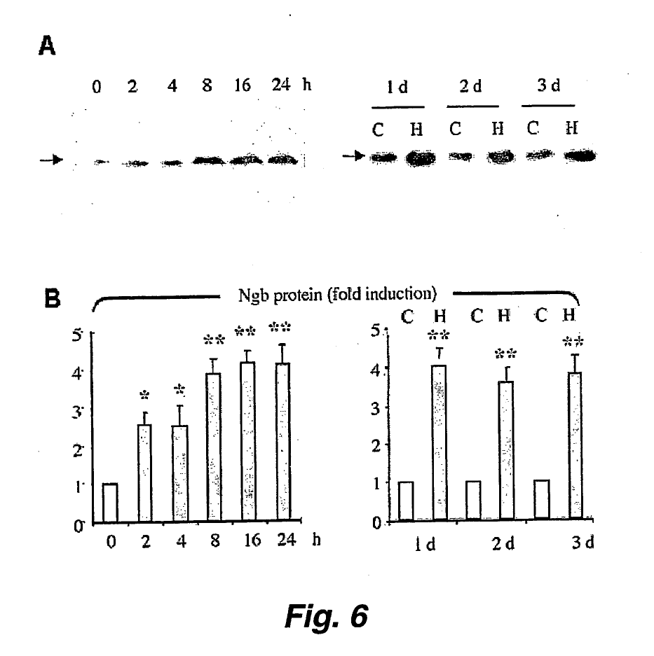

We next measured the expression of Ngb protein in HN33 cells treated with 50 μM hemin for up to 3 days. Western blot analysis showed that induction of Ngb protein was evident after 2 hours, persisted for at least 3 days, and reached about 4 times basal levels of expression FIG. 6).

Induction of Ngb Ex pression by Hemin, but not by Hypoxia, is Mediated through the sGC-PKG Signaling Pathway

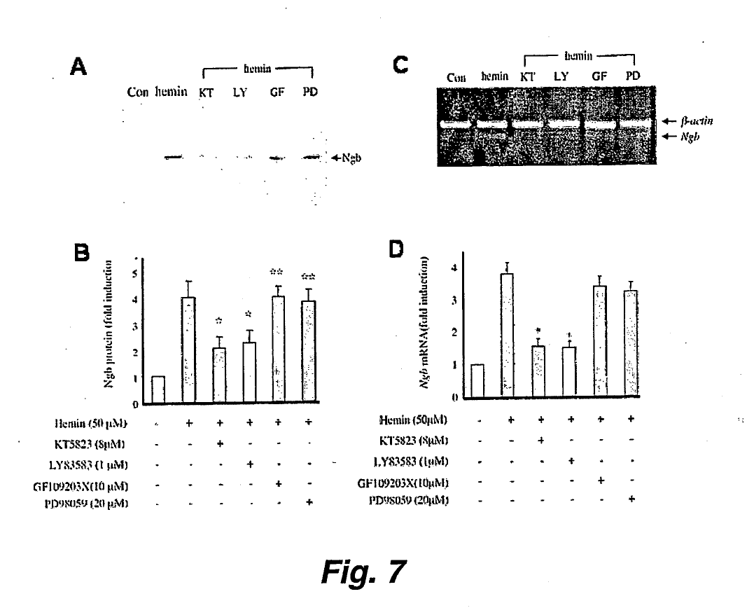

To investigate how hemin regulates the expression of Ngb in HN33 cells, we preincubated HN33 cells with the selective PKG inhibitor KT5823 (Gadbois et al. (1992) Proc Natl Acad Sci USA. 89:8626-8630), the sGC inhibitor LY83583 (Beasley et al. (1991) J Clin Invest., 87:602-608), the pan-spectrum PKC inhibitor GF109203X (Gould et al. (1995) Biochem J, 311:735-738), or the MEK1/2 inhibitor PD98059 (Id.). Western blot analysis showed that both the sGC inhibitor LY83583 (1 μM) and PKG inhibitor KT5823 (8 μM) significantly diminished induction of Ngb expression by hemin (FIG. 7). Quantitative RT-PCR showed that LY83583 and KT5823 also significantly inhibited Ngb expression at the mRNA level. In contrast, the PKC inhibitor GF109203X (10 μM) and the MEK1/2 inhibitor PD98059 (20 μM) had no significant effect. These results suggested that sGC and PKG are involved in hemin-induced Ngb expression.

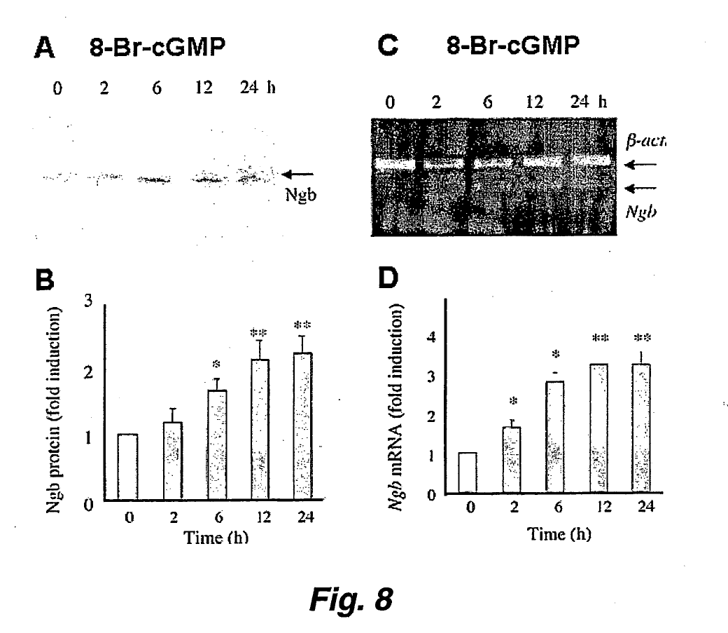

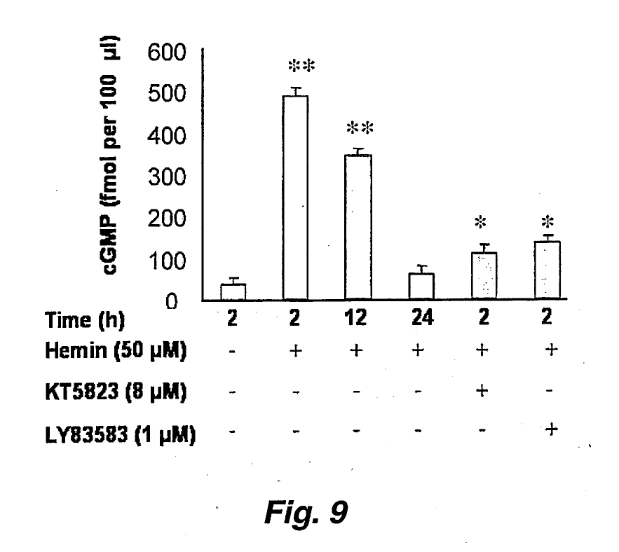

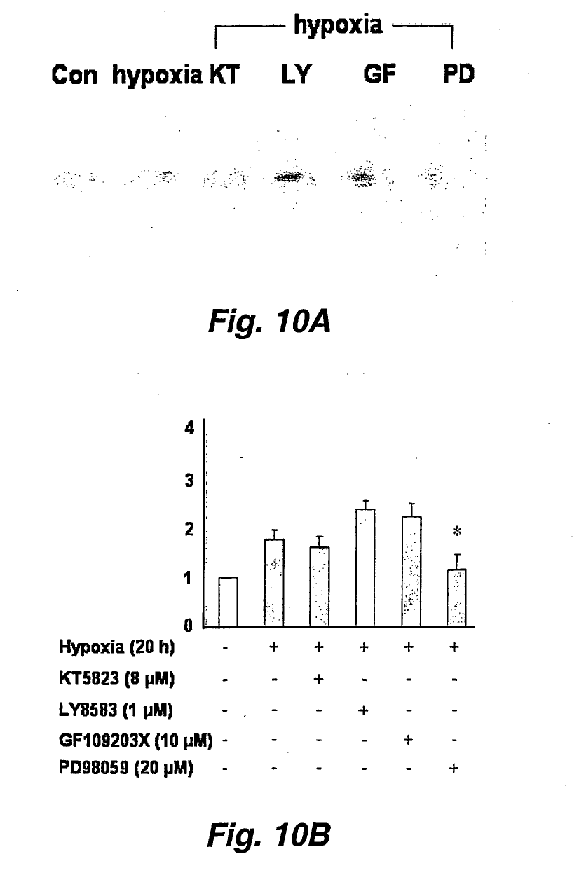

Next, HN33 cells were incubated with the cell membrane-permeant cGMP analog 8-Br-cGMP (10 μM), which activates PKG. As shown in FIG. 8, 8-Br-cGMP increased Ngb protein expression 2- to 2.5-fold, and Ngb mRNA expression 3- to 4-fold, in a time-dependent manner. To confirm that induction of Ngb expression by hemin is associated with an increase in cGMP levels, we measured intracellular cGMP in HN33 cells treated with 50 μM hemin. The results showed that cGMP levels were increased 8- to 15-fold after treatment with hemin for 2 to 12 hours (FIG. 9), which is consistent with the time course for induction of Ngb mRNA and protein. The fact that cGMP levels returned to near basal levels by 24 hours, whereas induction of Ngb persisted, suggests that cGMP synthesis is an early, transient step in the signaling pathway that leads to Ngb induction. Moreover, the ability of hemin to increase cGMP levels was abolished by both sGC and PKG inhibitors. Therefore, the sGC-PKG pathway may play a role in the induction of Ngb expression by hemin in neural cells.

Finally, we examined whether sGC/PKG signaling was also involved in the induction of Ngb expression by hypoxia. FIG. 7 shows that in contrast to the effect of hemin, hypoxic induction of Ngb was not blocked by LY83583 and KT5823, whereas it was blocked by the MEK inhibitor PD98059. Thus, distinct signaling mechanisms appear to be responsible for the effects of hemin and hypoxia on Ngb expression.

Discussion

Ngb is a recently identified vertebrate globin that is localized preferentially to cerebral neurons (Burmester et al. (2000) Nature, 407:520-523). Like hemoglobin and myoglobin, Ngb binds O₂, but little is known about its regulation or function. As illustrated in Example 1, neuronal Ngb expression is increased by hypoxia and by inducers of hypoxia-inducible factor-1α (such as CoCl₂ and deferoxamine) in vitro and by focal cerebral ischemia in vivo (Sun et al. (2001 Proc Natl Acad Sci USA. 98:15306-15311). Furthermore, hypoxic neuronal injury is increased by inhibiting Ngb expression with an antisense oligodeoxynucleotide (ODN) and reduced by Ngb overexpression. However, additional mechanisms for regulating Ngb expression are likely to exist.

Like other globins found in vertebrates, neuroglobin is a heme protein carrying a porphyrin ring with a central iron atom (Burmester et al. (2000) Nature, 407:520-523). Evidence has shown that hemoglobin and myoglobin can be induced by hemin (Rutherford et al. (1979) Nature, 280:164-165; Graber et al. J Biol Chem., 261:9150-9154). In this study, we demonstrated that Ngb can also be induced by hemin, and that this occurs in a dose- and time-dependent manner, at both the MRNA and protein levels. We demonstrated further that induction of Ngb expression by hemin appears to be mediated by the sGC-PKG pathway.

Heme is a prosthetic group of hemoproteins that include hemoglobin, catalase, and the cytochromes. As a prosthetic group, heme can regulate both the structure and the activity of hemoproteins, and has effects on gene expression involving both transcriptional and posttranscriptional events. The effect of heme on hemoglobin expression has been well studied. Heme increases hemoglobin production in K562 cells and in immature cultured erythroid cells through its effects on transcription, translation and assembly (Fibach et al. (1995) Blood 85:2967-2974; Ponka (1999) Am J Med Sci., 318:241-256) In addition to its effect on hemoglobin, treatment of K562 cells with hemin also up-regulates mRNA accumulation and protein expression of another erythroid-specific gene, the Kell-Cellano blood group antigen, KEL (Belhacene et al. (1998) FASEB J 12:531-539). Using mRNA differential display, hemin has also been shown to regulate genes expressed in early stages of K562 cell differentiation, such as the 62-kDa GAP-associated tyrosine phosphoprotein p62/SAM68, histone H2A.Z, the chaperonin Tcp20, and RIBB, a small G-protein of the Ras family (Zhu and Zhang (1999) Biochem Biophys Res Commmun., 258:87-93).

In neurons, hemin has neurotrophic effects that promote survival and rapid neurite outgrowth in cultured neuroblastoma cells and in neurons derived from the neural crest (Ishii et al. (1978) Nature, 274:372-374; Bonyhady et al. (1982) Dev Neurosci., 5:125-129). Direct administration of hemin to rats after transient forebrain ischemia is neuroprotective, as it significantly increases the number of viable neurons in cerebral cortex and striatum (Takizawa et al. (1998) J Cereb Blood Flow Metab., 18:559-569).

Induction of gene expression by hemin appears to involve several signal transduction pathways. In K562 and HEL cells, hemin induces hemoglobin expression by enhancing the activity of extracellular signal-regulated kinase 1/2 and inhibiting the activity of PKC (Woessmann et al. (2001) Exp Cell Res., 264:193-200; Yumoto et al. (1990) J Cell Physiol., 143:243-250). Recently, Ikuta et al proposed a model in which the sGC-PKG pathway mediates the effect of fetal hemoglobin-inducing agents, including hemin, in stimulating γ-globin gene expression (Ikuta et al. (2001) Proc Natl Acad Sci USA, 98:1847-1852). In this study, we showed that up-regulation of Ngb expression by hemin in HN33 neural cells was suppressed by sGC and PKG inhibitors, suggesting that this pathway is involved in heme-induced up-regulation of Ngb. In support of this hypothesis, hemin increased cGMP levels, and 8-Br-cGMP induced Ngb expression. We showed previously that Ngb expression in cortical neurons and HN33 cells is stimulated by hypoxia and by chemical inducers of hypoxia-inducible factor-1α, and that hypoxia-inducible Ngb expression helps promote neuronal survival from hypoxic injury (Sun et al. (2001 Proc Natl Acad Sci USA. 98:15306-15311). However, the induction of Ngb expression by hypoxia, appears to involve MEK rather than sGC/PKG. Of interest, both hemin and 8-Br-cGMP suppress, rather than enhance, the hypoxic induction of another hypoxia-inducible protein, vascular endothelial growth factor, in aortic smooth muscle cells (Liu et al. (1998) J Biol Chem., 273:15257-15262).

This study demonstrates that Ngb is a hemin-inducible gene, and that induction is regulated by the sGC-PKG pathway. Further characterization of this and other mechanisms that regulate Ngb expression should facilitate our understanding of Ngb function.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of screening for an agent that promotes neuronal survival from hypoxic-ischemic insult, said method comprising:
contacting a cell with a test agent;
and detecting the expression or activity of neuroglobin (Ngb) where an increase in neuroglobin expression or activity, as compared to the expression or activity of neuroglobin in a control indicates that said test agent is an agent promotes neuronal survival during or after hypoxic ischemic insult.

2. The method of claim 1, wherein said cell is a neural cell.

3. The method of claim 1, wherein said control comprises a neural cell contacted with said test agent at a lower concentration.

4. The method of claim 1, wherein said control comprises a cell that is not contacted with said test agent.

5. The method of claim 1, wherein the expression of Ngb is detected by detecting Ngb mRNA from said cell.

6. The method of claim 5, wherein said level of Ngb mRNA is measured by hybridizing said mRNA to a probe that specifically hybridizes to an Ngb nucleic acid.

7. The method of claim 6, wherein, wherein said hybridizing is according to a method selected from the group consisting of a Northern blot, a Southern blot using DNA derived from an Ngb RNA, an array hybridization, an affinity chromatography, and an in situ hybridization.

8. The method of claim 6, wherein said probe is a member of a plurality of probes that forms an array of probes.

9. The method of claim 5, wherein the level of Ngb mRNA is measured using a nucleic acid amplification reaction.

10. The method of claim 1, wherein the amount of Ngb gene product is detected by detecting the level of a neuroglobin (Ngb) protein from said cell.

11. The method of claim 10, wherein said detecting is via a method selected from the group consisting of capillary electrophoresis, a Western blot, mass spectroscopy, ELISA, immunochromatography, and immunohistochemistry.

12. The method of claim 1, wherein said cell is cultured ex vivo.

13. The method of claim 1, wherein said test agent is administered to an animal comprising a cell containing an Ngb nucleic acid or an Ngb protein.

14. The method of claim 1, wherein said test agent is administered to a brain section in culture.

15. A method of screening for an agent that promotes neuronal survival from hypoxic-ischemic insult, said method comprising:
providing a cell comprising an neuroglobin promoter and a reporter gene operably linked to said promoter;
contacting said cell with a test agent;
and detecting the expression or activity of said reporter gene where an increase in reporter gene, as compared to the expression or activity of the reporter gene in a control indicates that said test agent is an agent promotes neuronal survival during or after hypoxic ischemic insult.

16. The method of claim 15, wherein said reporter gene is selected from the group consisting of chloramphenicol acetyl transferase (CAT), luciferase, β-galactosidase (β-gal), alkaline phosphatase, horse radish peroxidase (HRP), growth hormone (GH), and green fluorescent protein (GFP).

17. A method of prescreening for an agent that promoting neuronal survival from hypoxic-ischemic insult, said method comprising:
i) contacting an Ngb nucleic acid or an Ngb protein with a test agent; and
ii) detecting specific binding of said test agent to said Ngb nucleic acid or protein.

18. The method of claim 17, further comprising recording test agents that specifically bind to said Ngb nucleic acid or protein in a database of candidate agents that promoting neuronal survival from hypoxic-ischemic insult.

19. The method of claim 17, wherein said test agent is not an antibody.

20. The method of claim 17, wherein said test agent is not a protein.

21. The method of claim 17, wherein said test agent is not a nucleic acid.

22. The method of claim 17, wherein said test agent is a small organic molecule.

23. The method of claim 17, wherein said detecting comprises detecting specific binding of said test agent to said Ngb nucleic acid.

24. The method of claim 23, wherein said binding is detected using a method selected from the group consisting of a Northern blot, a Southern blot using DNA derived from a Ngb RNA, an array hybridization, an affinity chromatography, and an in situ hybridization.

25. The method of claim 17, wherein said detecting comprises detecting specific binding of said test agent to said Ngb protein.

26. The method of claim 25, wherein said detecting is via a method selected from the group consisting of capillary electrophoresis, a Western blot, mass spectroscopy, ELISA, immunochromatography, and immunohistochemistry.

27. The method of claim 17, wherein said test agent is contacted directly to the Ngb nucleic acid or to the Ngb protein.

28. The method of claim 17, wherein said test agent is contacted to a cell containing the Ngb nucleic acid or the Ngb protein.

29. The method of claim 28, wherein said cell is cultured ex vivo.

30. The method of claim 17, wherein said test agent is contacted to an animal comprising a cell containing the Ngb nucleic acid or the Ngb protein.

31. A method of identifying a predilection to neural damage during a hypoxic or ischemic event in a mammal, said method comprising:
obtaining a biological sample from said mammal; and
detecting a mutation in an Ngb gene or gene product from said biological sample, where the presence of said mutation indicates a predilection to neural damage resulting from hypoxia or an ischemic event.

32. The method of claim 31, wherein said mutation is selected from the group consisting of an insertion, a deletion, a missense point mutation, and a nonsense point mutation.

33. The method of claim 31, wherein said detecting is by a method selected from the group consisting a Southern blot, a DNA amplification, comparative genomic hybridization, immunohistochemistry, and cytogenetics.

34. The method of claim 31, wherein said detecting comprises detecting a mutation in a polypeptide.

35. The method of claim 34, wherein said detecting comprises a method selected from the group consisting of capillary electrophoresis, a Western blot, mass spectroscopy, ELISA, immunochromatography, and immunohistochemistry.

36. A method of promoting neuronal survival from hypoxic ischemic insult, said method comprising modulating the concentration and/or activity of an Ngb gene product in a neural cell of a mammal.

37. The method of claim 36, wherein said modulating the concentration or activity of Ngb gene product comprises upregulating or repressing expression of a heterologous Ngb nucleic acid.

38. The method of claim 36, wherein said modulating comprises upregulating or repressing expression of an endogenous Ngb gene.

39. The method of claim 36, wherein said modulating comprises transfecting said cell with a vector that expresses an Ngb protein.

40. The method of claim 39, wherein said vector constitutively expresses an Ngb protein.

41. The method of claim 39, wherein expression of an Ngb protein by said vector is inducible.

42. A method of mitigating neurological damage associated with ischemia in a mammal, said method comprising increasing hemin levels or upregulating hemin expression in said mammal.

43. The method of claim 42, wherein said mammal is a human.

44. The method of claim 46, suffering from a condition selected from the group consisting of ischemia caused by myocardial infarction, stroke induced neuron death, reperfusion injury, traumatic head injury, cardiac arrest, and asphyxiation.

45. The method of claim 42, wherein said mammal is a non-human mammal.

46. A method of upregulating neuroglobin (NGB) expression in a mammal, said method comprising increasing hemin levels or upregulating hemin expression in said mamma.

47. A method of modulating neuroglobin expression, said method comprising modulating expression or activity of one or more components of the sGC-PKG pathway.
